(19) United States
(12) Patent Application Publication
(10) Pub. No.: US 2025/0041255 A1
(43) Pub. Date: Feb. 6, 2025

(54) COMPOSITIONS AND METHODS FOR TREATING AUTOIMMUNE SKIN DISEASES

(71) Applicant: Yale University, New Haven, CT (US)

(72) Inventors: Alicia Little, Orange, CT (US); Ping-Min Chen, Taipei City (TW); Joseph Craft, Hamden, CT (US)

(21) Appl. No.: 18/785,392

(22) Filed: Jul. 26, 2024

Related U.S. Application Data
(60) Provisional application No. 63/516,446, filed on Jul. 28, 2023.

(51) Int. Cl. A61K 31/195; A61K 38/12; A61P 37/06

(52) U.S. Cl. CPC A61K 31/195; A61K 38/12; A61P 37/06

(57) ABSTRACT

Methods of treating autoimmune skin diseases and disorders are provided. The methods typically include administrating to a subject with an autoimmune skin disease or disorder an effective amount of a hypoxia-inducible factor-1 (HIF-1) inhibitor. In preferred embodiments, the autoimmune disease is mediated at least in-part by T cells, particularly skin-infiltrating T cells. In some embodiments, the subject has cutaneous lupus (e.g., discoid cutaneous lupus, subacute cutaneous lupus, acute cutaneous lupus), pemphigus, pemphigoid, epidermolysis bullosa acquisita, vitiligo, lichen planus, lichen sclerosus, dermatomyositis, alopecia areata, or Sjögren's syndrome. HIF-1 inhibitors and pharmaceutical compositions including the same for use in the disclosed methods are also provided. The HIF-1 inhibitor can be, for example, a small molecule, functional nucleic acid, or inhibitory polypeptide or protein. The pharmaceutical composition can be formulated to suitable for the type and mode of administration, e.g., systemically or locally to skin effected by the autoimmune disease.

### CROSS-REFERENCE TO RELATED APPLICATIONS

This application claims benefit of and priority to U.S. Provisional Application No. 63/516,446, filed Jul. 28, 2023, the content of which is hereby incorporated herein by reference in its entirety.

### STATEMENT REGARDING FEDERALLY SPONSORED RESEARCH

This invention was made with government support under AR040072, AR074545 and AI152443 awarded by the National Institutes of Health. The government has certain rights in the invention.

### REFERENCE TO SEQUENCE LISTING

The Sequence Listing XML submitted as a file named “YU_8731_US_ST26.xml”, created on Jul. 24, 2024, and having a size of 21,870 bytes is hereby incorporated by reference pursuant to 37 C.F.R. § 1.834(c)(1).

### FIELD OF THE INVENTION

The disclosed invention is generally in the field of treatment of autoimmune skin diseases and specifically in the area of cutaneous lupus.

### BACKGROUND OF THE INVENTION

Cutaneous lupus erythematosus (CLE) may occur as isolated skin disease or may develop in the setting of SLE (5). Similar to the inflammatory infiltrate of lupus nephritis, the CLE inflammatory infiltrate is predominantly composed of T cells (6). How these changes contribute to disease pathogenesis is not known.

Thus, there remains a need to investigate phenotypic changes in skin-infiltrating T cells in CLE and the etiology thereof.

It is therefore an object of the invention to characterize the inflammatory infiltrate of T cells in cutaneous lupus.

It is a further object of the invention to provide compositions and methods of use therefore for treatment of cutaneous lupus and other skin autoimmune diseases.

### BRIEF SUMMARY OF THE INVENTION

Methods of treating autoimmune skin diseases and disorders are provided. The methods typically include administrating to a subject with an autoimmune skin disease or disorder an effective amount of a hypoxia-inducible factor-1 (HIF-1) inhibitor. In preferred embodiments, the autoimmune disease is mediated at least in-part by T cells, particularly skin-infiltrating T cells. In some embodiments, the HIF-1 inhibitor is administered in an effective amount to reduce the HIF-1 expression signature, reduce the cytotoxic activity, reduce expression of exhaustion marker(s), reduce Th17 phenotype, and/or reduce intracellular granzyme B in CD4+ and/or CD8+ T cells.

In some embodiments, the subject does not have one or more of lupus nephritis, cancer, or an angiogenic disease or disorder. In some embodiments, the subject does not have one or more of psoriasis, scleroderma, or pyogenic granulomas.

In some embodiments, the subject has cutaneous lupus (e.g., discoid cutaneous lupus, subacute cutaneous lupus, acute cutaneous lupus), pemphigus, pemphigoid, epidermolysis bullosa acquisita, vitiligo, lichen planus, lichen sclerosus, dermatomyositis, alopecia areata, or Sjögren's syndrome. In some embodiments, the subject has systemic lupus erythematosus (SLE). In other embodiments, the subject does not have SLE.

HIF-1 inhibitors and pharmaceutical compositions including the same for use in the disclosed methods are also provided. The HIF-1 inhibitor can be, for example, a small molecule, functional nucleic acid, or inhibitory polypeptide or protein. Exemplary HIF-1 inhibitors include PX-478, chemotin, topotecan, 103D5R, YC-1, GL331, geldanamycin, 2-ME2, bisphenol, berberine, PX-12, and pharmaceutically acceptable salts thereof. Functional nucleic acid HIF-1 inhibitors include antisense molecules, siRNA, miRNA, aptamers, ribozymes, RNAi, and external guide sequences, and can, for example target a segment of a nucleic acid encoding the amino acid sequence of SEQ ID NO:1, or the complement thereof, or variants thereof having a nucleic acid sequence at least 65%, 70%, 75%, 80%, 85%, 90%, or 95% identical to a nucleic acid encoding the amino acid sequence of SEQ ID NO:1, such as a segment of the nucleic acid sequence of SEQ ID NO:2, or the complement thereof, or a genomic sequence corresponding therewith, or variants thereof having a nucleic acid sequence at least 65%, 70%, 75%, 80%, 85%, 90%, or 95% identical to the nucleic acid sequence of SEQ ID NO:2.

HIF-1 inhibitory polypeptides can have, for example, the amino acid sequence of any of SEQ ID NOS:5-8, or variant thereof having a nucleic acid sequence at least 65%, 70%, 75%, 80%, 85%, 90%, or 95% identical to SEQ ID NOS:5-8.

Any of the HIF-1 inhibitors can further include a protein transduction domain.

Pharmaceutical compositions are provided and typically include a carrier. The pharmaceutical composition can be formulated to be suitable for the type and mode of administration, e.g., systemically or locally to skin affected by the autoimmune disease.

In a particular embodiment, the HIF-1 inhibitor is administered by topical administration, injection, or intralesional administration. In a more specific embodiments, the HIF-1 inhibitor is administered by topical administration in a pharmaceutical composition suitable for topical administration. Topical formulations can include a penetration enhancer.

Combination therapies are also provided, and may include administration of one or more additional active agents or other traditional therapies for treatment of the autoimmune skin disease or disorder.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims.

It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIGS. 1A-1F show that skin-infiltrating T cells show dominant HIF-1 transcript and protein signatures. FIG. 1A is a heatmap indicating differential expression of cytotoxic T, Th17, and Th1 cell markers between CD4⁺ and CD8⁺ skin-infiltrating and splenic T cells in a cutaneous lupus mouse model (MRL/lpr). Data show 2 or 3 biological replicates. FIG. 1B is a gene set enrichment analysis (GSEA) plots comparing gene signatures of skin-infiltrating to splenic CD4⁺ T cells based upon the hypoxia signature generated by comparing triple PHD-knockout CD4⁺ to wild type CD4⁺ T cells (17). FIG. 1C is a GSEA plot comparing gene signatures of skin-infiltrating to splenic CD8⁺ T cells based upon the hypoxia signature generated by comparing VHL tumor suppressor knockout CD8⁺ T cells to lymphocytic choriomeningitis virus specific P14 TCR transgenic CD8⁺ T cells taken from virally-infected mice (9). FIGS. 1D and 1E are representative data and summary of HIF1α staining of splenic vs skin-infiltrating CD4⁺ and CD8⁺ T cells isolated from the skin of 20- to 22-week-old MRL/lpr mice. FIG. 1F is a bar graph of Pimonidazole staining of splenic vs skin-infiltrating CD4⁺ and CD8⁺ T cells isolated from the skin of 20- to 22-week-old MRL/lpr mice. Representative of two experiments, n=6-7 animals per group. Data shown are mean±SEM; statistical analysis by two-tailed paired t-test (E, F). **** p<0.0001.

FIGS. 2A-2E show that skin-infiltrating T cells demonstrate activated cytotoxic and Th17 phenotypes. FIG. 2A is a bar graph of percent positive (left) and MFI (right) of granzyme B (GZB) of activated (CD44^{hi}) skin-infiltrating vs splenic CD4⁺ and CD8⁺ T cells isolated from 20- to 22-week-old MRL/lpr mice after stimulation with PMA/ionomycin for 4 hours post-isolation. FIG. 2B is a bar graph showing percentage of IFNγ⁺ cells of activated (CD44^{hi}) skin-infiltrating or splenic CD4⁺ and CD8⁺ T cells after stimulation as in (2A). FIG. 2C is a bar graph showing percentage of IL17⁺ cells of activated (CD44^{hi}) skin-infiltrating or splenic CD4⁺ and CD8⁺ T cells after stimulation as in (2A). FIG. 2D is a bar graph showing percentage of RORγt⁺ cells of activated (CD44^{hi}) skin-infiltrating vs splenic CD4⁺ and CD8⁺ T cells isolated as in (2A) FIG. 2E is a bar graph showing percentage of CCR6⁺ cells of activated (CD44^{hi}) skin-infiltrating vs splenic CD4⁺ and CD8⁺ T cells isolated as in (2A). n=8-10 mice in 2 experiments. Data are mean±SEM; statistical analysis by two-tailed paired t-test. ** p<0.01, * p<0.001, **** p<0.0001.

FIGS. 3A-3D show that HIF1α inhibition abrogates cutaneous disease and reduces cytotoxic activity in diseased MRL/lpr skin. FIG. 3A is a dot plot of Dermatitis scores in 20-week-old MRL/lpr mice after 4 weeks of treatment with either PX-478 or PBS (n=10 for each group). FIG. 3B is a dot plot of Histopathology damage score (see Methods) of diseased interscapular skin at age 20 weeks for mice treated as in (3A) (n=10 for each group). FIG. 3C is a dot plot showing association between clinical disease (dermatitis score) and percent Gzmb⁺ cells per high power field by RNA in situ hybridization in 20-week-old MRL/lpr mice treated as in (FIG. 3A). FIG. 3D is a bar graph quantification of percent Gzmb⁺ cells per high power field by RNA in situ hybridization in 20-week-old MRL/lpr mice treated as in (FIG. 3A) (3 high-power fields per mouse, n=10 mice for each group). Data shown are mean±SEM; statistical analysis by two-tailed Mann-Whitney test (FIGS. 3B, 3C), Spearman correlation (FIG. 3D), or nested two-tailed unpaired t-test (3F). * p<0.05, ** p<0.01, *** p<0.001.

FIGS. 4A-4E show that HIF1α inhibition reduces Hif1a and cytotoxic activity in CD8⁺ T cells isolated from diseased MRL/lpr skin. FIG. 4A is a combined UMAP plot of single-cell RNA sequencing data derived from cells isolated from the skin or spleen of 20-week-old MRL/lpr mice after 5 days of treatment with either PBS (vehicle) or selective HIF1α inhibitor PX-478 (treated). Cluster identities were determined using the CIPR (cluster identity predictor) package in R and confirmed by manual review of cell-type specific transcripts by cluster. (see Methods) FIG. 4B is a Dot plot demonstrating normalized expression level (average expression) and percent of cells expressing selected genes in CD4⁺ (top) or CD8⁺ (bottom) T cell clusters, separated by organ of origin (skin vs spleen) and treatment group (PBS vs PX-478). FIG. 4C is a violin plot showing the HIF1A enrichment score for CD8⁺ (left) or CD4⁺ (right) T cells, separated by organ of origin and treatment group. FIG. 4D is a violin plot showing the Cytotoxicity enrichment score for CD8⁺ T cells, separated by organ of origin and treatment group, as in (FIG. 4C). FIG. 4E is a violin plot showing the Th17 enrichment score for CD4⁺ T cells, separated by origin of cells and treatment group, as in (FIG. 4C). DNT, double-negative T cells. DC, dendritic cells. SPL, spleen. Pooled data from n=4 mice per treatment group. Statistical analysis by Kruskal-Wallis test. * p<0.05, ** p<0.01, *** p<0.001, **** p<0.0001.

FIGS. 5A-5D: Human DLE demonstrates expression of HIF1α- and cytotoxic molecules including granzyme B in the DLE lymphocytic infiltrate. FIG. 5A is a normalized HIF1A expression per region of interest (ROI) in DLE or normal control skin (n=3 patients, 3 ROIs per patient) as characterized by Nanostring GeoMx Digital Spatial Profiling. ROIs were selected to include T cell rich infiltrate. FIG. 5B is a Volcano plot of Nanostring DSP transcripts significantly (p<0.05) upregulated (orange), down-regulated (cyan) or not different (black, p>0.05) in DLE vs healthy control skin. Genes of interest are highlighted in purple. FIG. 5C is a GSEA comparing gene signatures from 6 DLE to 14 healthy skin samples (GSE109248) (25) based on the cytotoxicity signature generated from comparison of blister fluid to PBMCs in patients with Stevens-Johnson syndrome/toxic epidermal necrolysis (26). FIG. 5D is a bar graph of representative data and summary of granzyme B RNA ISH in FFPE DLE and healthy skin detected by RNAscope-RED Assay™ 200× and 400× original magnification (top, middle panels). Quantified with QuPath (lower panel) (n=5 DLE, 2 healthy control). Statistical analysis by nested unpaired two-tailed t-test. * p<0.05

FIG. 6 is a series of scatter plots showing a gating strategy for skin-infiltrating CD4⁺ and CD8⁺ T cells. After defining single cell lymphocyte gates by forward and side scatter, T cell populations were determined using gates for live hematopoietic (CD45.1⁺) CD3⁺ TCRβ⁺ B220⁻ cells. Intravascularly (IV) injected CD45.1-PE was used to separate tissue-infiltrating from circulating CD4⁺ and CD8⁺ T cells.

FIGS. 7A-7F show 10× single-cell RNA sequencing identifies CD4⁺ and CD8⁺ T cells in MRL/lpr skin and spleen by transcript and surface protein expression. FIG. 7A is a combined UMAP plot of single-cell RNA sequencing data derived from cells isolated from the skin or spleen of 20-week-old MRL/lpr mice after 5 days of treatment with either PBS (vehicle) or selective HIF1α inhibitor PX-478 (treated), colored by organ of origin (skin vs spleen) and treatment group (PBS vs PX-478). FIG. 7B is a combined UMAP plot shaded to demonstrate normalized expression level of Cd3e, which highlights clusters identified as T cells (0, 2, 3, 4, 5, 9, 26) for further analysis. Cluster identities were determined using the CIPR (cluster identity predictor) package in R and confirmed by manual review of cell-type specific transcripts by cluster (see Methods). FIG. 7C is a violin plot demonstrating Cd3e, Cd4, and Cd8a transcript expression by cluster, highlighting clusters identified as CD4⁺ T cells (0, 5, 9), CD8⁺ T cells (2, 26) and double-negative T cells (3, 4). FIG. 7D is a combined UMAP plots shaded to demonstrate normalized expression level of CD8A protein (left) or transcript (right). FIG. 7E is combined UMAP plots shaded to demonstrate normalized expression level of CD4 protein (left) and transcript (right). FIG. 7F is a Dot plot demonstrating normalized expression level (average expression) and percent of cells expressing selected exhaustion-associated genes in CD4⁺ (top) or CD8⁺ (bottom) T cell clusters, separated by organ of origin (skin vs spleen) and treatment group (PBS vs PX-478). SPL, spleen.

FIGS. 8A and 8B: Human DLE demonstrates expression of cytotoxic molecules including granzyme B in the DLE lymphocytic infiltrate in proximity to the dermoepidermal junction and hair follicle. FIGS. 8A and 8B are line graphs showing the percentage of GZMB+ CD3⁺ T cells as a function of distance from the dermoepidermal junction (FIG. 8A) or hair follicle (FIG. 8B) as detected in FFPE DLE skin by RNAscope Multiplex Fluorescent V2 Assay™ at 400× original magnification. Quantified with HALO (see Methods).

### DETAILED DESCRIPTION OF THE INVENTION

The disclosed method and compositions can be understood more readily by reference to the following detailed description of particular embodiments and the Example included therein and to the Figures and their previous and following description.

### I. Definitions

“Treatment” or “treating” means to administer a composition to a subject or a system with an undesired condition (e.g., autoimmune disease). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition (e.g., autoimmune disease). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the term “carrier” or “excipient” refers to an organic or inorganic ingredient, natural or synthetic inactive ingredient in a formulation, with which one or more active ingredients are combined.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “inhibiting” as used here in relation to HIF means preventing, reducing, or otherwise ameliorating HIF production or activation. For example, depending on the circumstances, including nature of the condition being treated, it may not be necessary that inhibition should mean completely blocking HIF production or activation, but reducing HIF production or activation to a sufficient degree to enable the desired effect to be achieved.

As used herein, the term “topically active agents” refers to compositions of the present disclosure that elicit pharmacological responses at the site of application (contact in a topical application) to a host.

As used herein, the term “topically” refers to application of the compositions of the present disclosure to the surface of the skin and mucosal cells and tissues.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. Preferably, the subject is a mammal. The mammal can be a human, non-human primate, mouse, rat, dog, cat, horse, or cow, but are not limited to these examples. Mammals other than humans can be advantageously used as subjects that represent animal models of HIF or hypoxia related pathologies. In addition, the methods described herein can be used to treat domesticated animals and/or pets. A subject can be male or female.

For simplicity, chemical moieties are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms “alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The term “halo” or “halogen” refers to any radical of fluorine, chlorine, bromine or iodine.

The term “acyl” refers to an alkylcarbonyl, alkoxycarbonyl, cycloalkylcarbonyl, arylcarbonyl, heterocyclylcarbonyl, or heteroarylcarbonyl substituent, any of which may be further substituted by substituents. Exemplary acyl groups include, but are not limited to, (C₁-C₆)alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions.

The term “alkyl” refers to saturated non-aromatic hydrocarbon chains that may be a straight chain or branched chain, containing the indicated number of carbon atoms (these include without limitation propyl, allyl, or propargyl), which may be optionally inserted with N, O, or S. For example, C₁-C₆ indicates that the group may have from 1 to 6 (inclusive) carbon atoms in it.

The term “alkenyl” refers to an alkyl that comprises at least one double bond. Exemplary alkenyl groups include, but are not limited to, for example, ethenyl, propenyl, butenyl, 1-methyl-2-buten-1-yl and the like.

The term “alkynyl” refers to an alkyl that comprises at least one triple bond.

The term “alkoxy” refers to an —O-alkyl radical.

The term “aminoalkyl” refers to an alkyl substituted with an amino.

The term “mercapto” refers to an SH radical.

The term “thioalkoxy” refers to an —S-alkyl radical.

The term “aryl” refers to monocyclic, bicyclic, or tricyclic aromatic ring system wherein 0, 1, 2, 3, or 4 atoms of each ring may be substituted by a substituent. Exemplary aryl groups include, but are not limited to, phenyl, naphthyl, anthracenyl, azulenyl, fluorenyl, indanyl, indenyl, naphthyl, phenyl, tetrahydronaphthyl, and the like.

The term “arylalkyl” refers to alkyl substituted with an aryl.

The term “cyclyl” or “cycloalkyl” refers to saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Exemplary cycloalkyl groups include, but are not limited to, cyclopropyl, cyclobutyl, cyclopentyl, cyclopentenyl, cyclohexyl, cyclohexenyl, cycloheptyl, cyclooctyl, and the like.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, and the like.

The term “heteroarylalkyl” refers to an alkyl substituted with a heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Exemplary heterocyclyl groups include, but are not limited to piperazinyl, pyrrolidinyl, dioxanyl, morpholinyl, tetrahydrofuranyl, and the like.

The term “haloalkyl” refers to an alkyl group having one, two, three or more halogen atoms attached thereto. Exemplary haloalkyl groups include, but are not limited to chloromethyl, bromoethyl, trifluoromethyl, and the like.

The term “optionally substituted” means that the specified group or moiety, such as an alkyl, alkoxy, aryl group, heteroaryl group and the like, is unsubstituted or is substituted with one or more (typically 1 to 4 substituents) independently selected from the group of substituents listed below in the definition for “substituents” or otherwise specified.

The term “substituents” refers to a group “substituted” on an alkyl, alkenyl, alkynyl, cycloalkyl, aryl, heterocyclyl, or heteroaryl group at any atom of that group. Suitable substituents include, without limitation, halo, hydroxy, oxo, nitro, haloalkyl, alkyl, alkenyl, alkynyl, alkaryl, aryl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

As used herein, the term “pharmaceutically acceptable salt” refers to salts of the compounds described herein which are substantially non-toxic to living organisms. Typical pharmaceutically acceptable salts include those salts prepared by reaction of the compounds of the present invention with a pharmaceutically acceptable mineral or organic acid or an organic or inorganic base. Such salts are known as acid addition and base addition salts.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, y-hydroxybutyrate, glycolate, tartrate, methanesulfonate, propanesulfonate, naphthalene-1-sulfonate, napththalene-2-sulfonate, mandelate and the like. Preferred pharmaceutically acceptable acid addition salts are those formed with mineral acids such as hydrochloric acid and hydrobromic acid, and those formed with organic acids such as maleic acid and methanesulfonic acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred. It should be recognized that the particular counterion forming a part of any salt of this invention is usually not of a critical nature, so long as the salt as a whole is pharmacologically acceptable and as long as the counterion does not contribute undesired qualities to the salt as a whole. It is further understood that the above salts may form hydrates or exist in a substantially anhydrous form.

The compounds described herein and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

As used herein, the terms “stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds described herein as suitable for use in the present invention which may give rise to stereoisomerism, the invention contemplates stereoisomers and mixtures thereof.

The term “enantiomers” means a pair of stereoisomers that are non-superimposable mirror images of each other.

The term “diastereoisomers” or “diastereomers” mean optical isomers which are not mirror images of each other.

The term “racemic mixture” or “racemate” mean a mixture containing equal parts of individual enantiomers.

The term “non-racemic mixture” means a mixture containing unequal parts of individual enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation: ee=100×(E¹−E²)/(E¹+E²), wherein E¹ is the amount of the first enantiomer and E² is the amount of the second enantiomer.

In some embodiments, compound described herein have an enantiomeric excess of at least 1%, 5%, 10%, 20%, 30%, 40%, 50%, 60%, 70%, 80%, 90%, 95% or more. Generally, an ee of greater than 90% is preferred, an ee of greater than 95% is most preferred and an ee of greater than 99% is most especially preferred.

Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art. In addition, the enantiomers of compounds can be resolved by one of ordinary skill in the art using standard techniques well known in the art, such as those described by J. Jacques, et al., “Enantiomers, Racemates, and Resolutions”, John Wiley and Sons, Inc., 1981. Examples of resolutions include recrystallization techniques or chiral chromatography.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

Every compound disclosed herein is intended to be and should be considered to be specifically disclosed herein. Further, every subgroup that can be identified within this disclosure is intended to be and should be considered to be specifically disclosed herein. As a result, it is specifically contemplated that any compound, or subgroup of compounds can be either specifically included for or excluded from use or included in or excluded from a list of compounds.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular polypeptide is disclosed and discussed and a number of modifications that can be made to a number of polypeptides are discussed, specifically contemplated is each and every combination and permutation of polypeptides and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### II. Methods of Use

The transcription factor hypoxia-inducible factor-1 (HIF-1) plays a critical role in the cellular and developmental response to hypoxia, as well as in response to inflammation-associated signals including T cell and Toll-like receptor and cytokine signaling (7, 8). HIF-1 is a heterodimer, composed of HIF-1α (tightly regulated at the transcript and protein levels) bound to HIF-1β (constitutively expressed). In T cells, HIF-1 enhances T cell effector function, including inflammatory cytokine and cytotoxic molecule production (4, 9, 10), and blocks terminal differentiation and exhaustion. HIF1 also targets Foxp3 for degradation (11), decreasing Treg differentiation and enhancing Th17 development (11, 12).

A canonical sequence for human HIF-1α is UniProt Accession No. Q16665-HIF1A HUMAN:

(SEQ ID NO: 1, Q16665. HIF1A_HUMAN)
MEGAGGANDKKKISSERRKEKSRDAARSRRSKESEVFYELAHQLPL
PHNVSSHLDKASVMRLTISYLRVRKLLDAGDLDIEDDMKAQMNCF
YLKALDGFVMVLTDDGDMIYISDNVNKYMGLTQFELTGHSVEDFT
HPCDHEEMREMLTHRNGLVKKGKEQNTQRSFFLRMKCTLTSRGRT
MNIKSATWKVLHCTGHIHVYDTNSNQPQCGYKKPPMTCLVLICEP
IPHPSNIEIPLDSKTELSRHSLDMKFSYCDERITELMGYEPEELL
GRSIYEYYHALDSDHLTKTHHDMFTKGQVTTGQYRMLAKRGGYVW
VETQATVIYNTKNSQPQCIVCVNYVVSGIIQHDLIFSLQQTECVL
KPVESSDMKMTQLFTKVESEDTSSLEDKLKKEPDALTLLAPAAGD
TIISLDFGSNDTETDDQQLEEVPLYNDVMLPSPNEKLQNINLAMS
PLPTAETPKPLRSSADPALNQEVALKLEPNPESLELSFTMPQIQD
QTPSPSDGSTROSSPEPNSPSEYCFYVDSDMVNEFKLELVEKLFA
EDTEAKNPESTQDTDLDLEMLAPYIPMDDDFQLRSFDQLSPLESS
SASPESASPQSTVTVFQQTQIQEPTANATTTTATTDELKTVTKDR
MEDIKILIASPSPTHIHKETTSATSSPYRDTQSRTASPNRAGKGV
IEQTEKSHPRSPNVLSVALSQRTTVPEEELNPKILALQNAQRKRK
MEHDGSLFQAVGIGTLLQQPDDHAATTSLSWKRVKGCKSSEQNGM
EQKTIILIPSDLACRLLGQSMDESGLPQLTSYDCEVNAPIQGSRN
LLQGEELLRALDQVN

An exemplary variant of canonical HIF-1α is Q16665-2, which differs from SEQ ID NO:1 by: 735-735: G→I, 736-826: missing.

Another exemplary variant of canonical HIF-1α is Q16665-3, which differs from SEQ ID NO:1 by: 1-12: MEGAGGANDKKK (SEQ ID NO:3)→MSSQCRSLENKFVFLKEGLGNSKPEELEEIRIENGR (SEQ ID NO:4) Nucleic acid sequences encoding HIF-1α are also known in the art, and include, for example, GenBank: U22431.1, Human hypoxia-inducible factor 1 alpha (HIF-1 alpha) mRNA, complete cds, which encode SEQ ID NO:1:

| | |
|---|---|
| 1 | gtgaagacat cgcggggacc gattcaccat ggagggcgcc ggcggcgcga acgacaagaa |
| 61 | aaagataagt tctgaacgtc gaaaagaaaa gtctcgagat gcagccagat ctcggcgaag |
| 121 | taaagaatct gaagtttttt atgagcttgc tcatcagttg ccacttccac ataatgtgag |
| 181 | ttcgcatctt gataaggcct ctgtgatgag gcttaccatc agctatttgc gtgtgaggaa |
| 241 | acttctggat gctggtgatt tggatattga agatgacatg aaagcacaga tgaattgctt |
| 301 | ttatttgaaa gccttggatg gttttgttat ggttctcaca gatgatggtg acatgattta |
| 361 | catttctgat aatgtgaaca aatacatggg attaactcag tttgaactaa ctggacacag |
| 421 | tgtgtttgat tttactcatc catgtgacca tgaggaaatg agagaaatgc ttacacacag |
| 481 | aaatggcctt gtgaaaaagg gtaaagaaca aaacacacag cgaagctttt ttctcagaat |
| 541 | gaagtgtacc ctaactagcc gaggaagaac tatgaacata aagtctgcaa catggaaggt |
| 601 | attgcactgc acaggccaca ttcacgtata tgataccaac agtaaccaac ctcagtgtgg |
| 661 | gtataagaaa ccacctatga cctgcttggt gctgatttgt gaacccattc ctcacccatc |
| 721 | aaatattgaa attcctttag atagcaagac tttcctcagt cgacacagcc tggatatgaa |
| 781 | attttcttat tgtgatgaaa gaattaccga attgatggga tatgagccag aagaactttt |
| 841 | aggccgctca atttatgaat attatcatgc tttggactct gatcatctga ccaaaactca |
| 901 | tcatgatatg tttactaaag gacaagtcac cacaggacag tacaggatgc ttgccaaaag |
| 961 | aggtggatat gtctgggttg aaactcaagc aactgtcata tataacacca agaattctca |
| 1021 | accacagtgc attgtatgtg tgaattacgt tgtgagtggt attattcagc acgacttgat |
| 1081 | tttctccctt caacaaacag aatgtgtcct taaaccggtt gaatcttcag atatgaaaat |
| 1141 | gactcagcta ttcaccaaag ttgaatcaga agatacaagt agcctctttg acaaacttaa |
| 1201 | gaaggaacct gatgctttaa ctttgctggc cccagccgct ggagacacaa tcatatcttt |
| 1261 | agattttggc agcaacgaca cagaaactga tgaccagcaa cttgaggaag taccattata |
| 1321 | taatgatgta atgctcccct cacccaacga aaaattacag aatataaatt tggcaatgtc |
| 1381 | tccattaccc accgctgaaa cgccaaagcc acttcgaagt agtgctgacc ctgcactcaa |
| 1441 | tcaagaagtt gcattaaaat tagaaccaaa tccagagtca ctggaacttt cttttaccat |
| 1501 | gccccagatt caggatcaga cacctagtcc ttccgatgga agcactagac aaagttcacc |
| 1561 | tgagcctaat agtcccagtg aatattgttt ttatgtggat agtgatatgg tcaatgaatt |
| 1621 | caagttggaa ttggtagaaa aactttttgc tgaagacaca gaagcaaaga acccattttc |
| 1681 | tactcaggac acagatttag acttggagat gttagctccc tatatcccaa tggatgatga |
| 1741 | cttccagtta cgttccttcg atcagttgtc accattagaa agcagttccg caagccctga |
| 1801 | aagcgcaagt cctcaaagca cagttacagt attccagcag actcaaatac aagaacctac |
| 1861 | tgctaatgcc accactacca ctgccaccac tgatgaatta aaaacagtga caaaagaccg |
| 1921 | tatggaagac attaaaatat tgattgcatc tccatctcct acccacatac ataaagaaac |
| 1981 | tactagtgcc acatcatcac catatagaga tactcaaagt cggacagcct caccaaacag |
| 2041 | agcaggaaaa ggagtcatag aacagacaga aaaatctcat ccaagaagcc ctaacgtgtt |
| 2101 | atctgtcgct ttgagtcaaa gaactacagt tcctgaggaa gaactaaatc caaagatact |
| 2161 | agctttgcag aatgctcaga gaaagcgaaa aatggaacat gatggttcac tttttcaagc |
| 2221 | agtaggaatt ggaacattat tacagcagcc agacgatcat gcagctacta catcactttc |
| 2281 | ttggaaacgt gtaaaaggat gcaaatctag tgaacagaat ggaatggagc aaaagacaat |
| 2341 | tattttaata ccctctgatt tagcatgtag actgctgggg caatcaatgg atgaaagtgg |
| 2401 | attaccacag ctgaccagtt atgattgtga agttaatgct cctatacaag gcagcagaaa |
| 2461 | cctactgcag ggtgaagaat tactcagagc tttggatcaa gttaactgag ctttttctta |
| 2521 | atttcattcc tttttttgga cactggtggc tcactaccta aagcagtcta tttatatttt |
| 2581 | ctacatctaa ttttagaagc ctggctacaa tactgcacaa acttggttag ttcaattttt |
| 2641 | gatccccttt ctacttaatt tacattaatg ctctttttta gtatgttctt taatgctgga |
| 2701 | tcacagacag ctcattttct cagttttttg gtatttaaac cattgcattg cagtagcatc |
| 2761 | attttaaaaa atgcaccttt ttatttattt atttttggct agggagttta tccctttttc |
| 2821 | gaattatttt taagaagatg ccaatataat ttttgtaaga aggcagtaac ctttcatcat |
| 2881 | gatcataggc agttgaaaaa tttttacacc ttttttttca cattttacat aaataataat |
| 2941 | gctttgccag cagtacgtgg tagccacaat tgcacaatat attttcttaa aaaataccag |
| 3001 | cagttactca tggaatatat tctgcgttta taaaactagt ttttaagaag aaattttttt |
| 3061 | tggcctatga aattgttaaa cctggaacat gacattgtta atcatataat aatgattctt |
| 3121 | aaatgctgta tggtttatta tttaaatggg taaagccatt tacataatat agaaagatat |
| 3181 | gcatatatct agaaggtatg tggcatttat ttggataaaa ttctcaattc agagaaatca |
| 3241 | tctgatgttt ctatagtcac tttgccagct caaaagaaaa caatacccta tgtagttgtg |
| 3301 | gaagtttatg ctaatattgt gtaactgata ttaaacctaa atgttctgcc taccctgttg |
| 3361 | gtataaagat attttgagca gactgtaaac aagaaaaaaa aaatcatgca ttcttagcaa |
| 3421 | aattgcctag tatgttaatt tgctcaaaat acaatgtttg attttatgca ctttgtcgct |
| 3481 | attaacatcc tttttttcat gtagatttca ataattgagt aattttagaa gcattatttt |
| 3541 | aggaatatat agttgtcaca gtaaatatct tgttttttct atgtacattg tacaaatttt |
| 3601 | tcattccttt tgctctttgt ggttggatct aacactaact gtattgtttt gttacatcaa |
| 3661 | ataaacatct tctgtgga |

(SEQ ID NO: 2, GenBank: U22431.1, Human hypoxia-inducible factor 1
alpha (HIF-1 alpha) mRNA, complete cds).

Under physiological conditions, the oxygen tension in healthy human skin ranges from physiologic (10%, or 76 mmHg) to severely hypoxic (0.1%, or 0.76 mmHg), with the lowest oxygen tension found in the epidermis and portions of some hair follicles and sebaceous glands (13, 14). In CLE, cellular infiltrates are predominantly located in the dermis (6, 15), which contains the dermal vasculature. In healthy skin, this region has physiologic oxygen tension. However, the local oxygen tension in lesional CLE skin is not known, and local tissue damage and inflammation in CLE skin may create a relatively hypoxic microenvironment. In addition, HIF-1 can be upregulated in a hypoxia-independent manner. HIF-1 is accumulated in skin in response to ultraviolet (UV) light, mediated by mitochondrial reactive oxygen species (16), and HIF-1 is also upregulated in response to inflammation-associated signals including T cell and Toll-like receptor and cytokine signaling (7).

The results presented in the Examples below show that skin-infiltrating CD4+ and CD8+ T cells also express high levels of HIF-1. Skin-infiltrating T cells demonstrate a strong cytotoxic signature at the transcript and protein level, and HIF-1 inhibition abrogates skin and systemic disease in association with decreased T cell cytotoxic activity. The results also demonstrate in human CLE tissue that the T cell rich inflammatory infiltrate exhibits increased amounts of HIF-1 and a cytotoxic signature. Granzyme B-expressing T cells are concentrated at sites of skin tissue damage in CLE, indicating relevance of this pathway to human disease.

Thus, compositions and methods for treating cutaneous lupus, and other skin autoimmune diseases are provided.

### A. Methods of Treatment

Typically, the methods include administering to a subject skin autoimmune disease, a composition including an effective amount of a HIF-1 inhibitor.

The HIF-1 inhibitor can be administered systemically, or locally, for example to the site of the skin autoimmune disease symptoms. Suitable formulations and routes of administration are discussed in more detail below. In some embodiments, the composition is administered orally. In other embodiments, the composition of the directly to the effected area, e.g., the skin, for example, by topical administration, patch application, injection (e.g., microinjection), intralesional administration, etc.

In some embodiments, the HIF-1 inhibitor is administered in an effective amount to reduce one or more symptoms the autoimmune skin disease. In some embodiments, the HIF-1 inhibitor is administered in an effective amount to reduce the HIF-1 expression signature in CD4+ and/or CD8+ T cells, most particularly skin-infiltrating CD4+ and/or CD8+ T cells (see, e.g., experiments below). In some embodiments, the HIF-1 inhibitor is administered in an effective amount to reduce the cytotoxic activity (e.g., decrease Gzmb, Gzmk, and/or Fasl transcripts), reduce exhaustion markers (e.g., decrease Pdcd1 and Tigit transcripts), and/or Th17 phenotype of CD4+ and/or CD8+ T cells, most particularly skin-infiltrating CD4+ and/or CD8+ T cells. In some embodiments, the HIF-1 inhibitor is administered in an effective amount to reduce intracellular granzyme B in CD4+ and/or CD8+ T cells, most particularly skin-infiltrating CD4+ and/or CD8+ T cells (e.g., compared to splenic T cells). The inhibitor can be, for example, an inhibitor of HIF-1α expression and/or activity, HIF-13 expression and/or activity, an interaction between HIF-1α and HIF-1β (i.e., heterodimer formation), or a combination thereof. Preferably, the inhibitor is an inhibitor of HIF-1α expression and/or activity, an interaction between HIF-1α and HIF-13 (i.e., heterodimer formation), or a combination thereof.

### B. Subjects to be Treated

The subjects to be treated typically have an autoimmune skin disease. Such diseases include, but are not limited to, cutaneous lupus, pemphigus, pemphigoid, epidermolysis bullosa acquisita, vitiligo, lichen planus, lichen sclerosus, dermatomyositis, alopecia areata, or Sjögren's syndrome. See, e.g., Autoimmunity: From Bench to Bedside [Internet], “Chapter 34 Dermatological autoimmune diseases,” Anaya J M, Shoenfeld Y, Rojas-Villarraga A, et al., editors. Bogota (Colombia): El Rosario University Press; 2013 Jul. 18.

In preferred embodiments, the subject has a cutaneous lupus. Cutaneous lupus is a type of lupus. It causes a red, scaly rash on the skin. There are three types of cutaneous (skin) lupus. Each type has similar symptoms, including a red, scaly rash that often results from exposure to the sun. See, e.g., McDaniel et al., “Discoid Lupus Erythematosus.” [Updated 2022 Oct. 30]. In: StatPearls [Internet]. Treasure Island (FL): StatPearls Publishing; 2023 January-. Available from ncbi.nlm.nih.gov/books/NBK493145/; and Cleveland Clinic website at “Cutaneous Lupus (Skin Lupus).”

In discoid cutaneous lupus the skin lesions are round (disk-shaped), thick, scaly and red, and may be accompanied by pain, itching and burning.

In subacute cutaneous lupus red borders develop around the edges of these lesions. They can look like a ring with a darker red circle on the ring's outer edge. They do not usually hurt, itch or scar.

Acute cutaneous lupus is characterized by a red rash that often develops along the cheeks and the bridge of the nose, usually after sun exposure. Providers call this a malar rash or “butterfly rash” because of its shape. This characteristic “butterfly rash” is a sign of systemic lupus.

In some embodiments, the subject has cutaneous lupus and another form of lupus. In some embodiments, the subject has cutaneous lupus, and does not have one or more other forms of lupus. In some embodiments, the subject does not have lupus nephritis. In some embodiments, the subject has systemic lupus erythematosus (SLE). In some embodiments, the subject does not have SLE. About 10% of all lupus cases are cutaneous and 65% of people with systemic lupus will develop skin lupus.

In some embodiments, the subject has another autoimmune skin disease. For example, scleroderma and morphea is a condition that causes the skin to become thick and rigid.

Pemphigus (e.g., Pemphigus Vulgaris (PV), Pemphigus Foliaceous, Pemphigus erythematosus (PE), Drug-induced pemphigus, IgA pemphigus, Pemphigus vegetans, Paraneoplastic pemphigus) is a group of blistering diseases with an organ-specific autoimmune pathogenesis that affects the skin and mucous membranes. The disease is characterized by blisters and erosions caused by intraepidermal cell detachment in a process termed acantholysis.

Bullous pemphigoid is a blistering autoimmune disease characterized by the separation of the dermal-epidermal junction (DEJ) accompanied by inflammatory cell infiltration in the upper dermis.

Epidermolysis bullosa acquisita (EBA) is a rare acquired sub-epidermal bullous disease that exhibits certain clinical similarities to the genetic forms of dystrophic epidermolysis bullosa. Autoimmune EBA affects the anchoring fibrils (AF) that anchor the BMZ to the dermis. The principal component of these fibrils is collagen VII (COL7) and patients develop anti-COL7 autoantibodies, causing skin fragility, erosions, and blisters.

Vitiligo is a chronic depigmentation disease that affects the melanocytes, and the destruction of the melanocytes is the central pathological event that causes the depigmentation. This pathology can be presented clinically as a primary disease or can be a component of multiple autoimmune processes such as thyroid disease, pernicious anemia, rheumatoid arthritis, lupus, adult onset autoimmune diabetes, and Addison's disease.

Lichen planus (LP) and the LP variants lichen planopilaris, lichen planus pigmentosus, hypertrophic LP, bullous LP, and mucosal LP, are a group of autoimmune skin diseases characterized by a band-like lymphocytic infiltrate in the dermis and vacuolar interface changes in the basal layer of the epidermis. These disorders may cause pruritic rashes on the skin, erosions in the mucosa, or destruction of the hair follicles. Lichen sclerosus (LS) is a similar disorder that is characterized by a band-like lymphocytic infiltrate and sclerosis of the dermis. LS affects genital skin more frequently than extra-genital skin, and often causes pruritus, scarring and may predispose to development of squamous cell carcinoma. Dermatomyositis is an autoimmune skin disease that can cause muscle inflammation and skin rashes. See, e.g., Tziotzios, et al., J Am Acad Dermatol. 2018 November; 79(5):789-804. doi: 10.1016/j.jaad.2018.02.010. PMID: 30318136.

Alopecia areata is a common, non-scarring hair loss disorder involving autoimmune destruction of the hair follicle that can result in patches of hair loss or can cause complete hair loss from the body (alopecia universalis). See, e.g., Strazzulla, et al., J Am Acad Dermatol. 2018 January; 78(1):1-12. doi: 10.1016/j.jaad.2017.04.1141. PMID: 29241771.

Sjögren's syndrome a chronic (long-lasting) autoimmune disorder that happens when the immune system attacks the glands that make moisture in the eyes, mouth, and other parts of the body, leading to dry mouth, dry eyes, and sometimes dry skin.

Tumor hypoxia increases malignant progression and metastasis by promoting angiogenesis through the induction of proangiogenic proteins such as VEGF (Schweiki, D. et al. Vascular endothelial growth factor induced by hypoxia may mediate hypoxia-induced angiogenesis. Nature 359, 843-5 (1992)). Thus, it is believed that inhibition of HIF-mediated gene regulation can reduce tumor angiogenesis and prevent the adaptive metabolic response to hypoxia, thus suppressing tumor growth. In some embodiments, the subject does not have cancer and/or an angiogenic disease or disorder (e.g., a disease, disorder, or condition characterized or caused by aberrant or unwanted, e.g., stimulated or suppressed, formation of blood vessels (angiogenesis)).

In particular embodiments, the subject does not have one or more of psoriasis, scleroderma, and pyogenic granulomas.

### C. Combination Therapy

HIF-1 inhibitors can be administered to a subject alone, or in combination with one or more additional active agents or other treatment strategies, particularly those for the treatment of autoimmune skin disorders such as cutaneous lupus.

In some embodiments, the HIF-1 inhibitor is administered in combination with instructions to avoiding the sun and fluorescent light. Lupus skin lesions are extremely sensitive to natural and artificial light

In some embodiments, the HIF-1 inhibitor is administered in combination with a corticosteroid medication directly into the effected area. Steroid injections reduce inflammation. You may need these injections every few weeks.

In some embodiments, the HIF-1 inhibitor is administered in combination with an immune modulator or suppressant such as hydroxychloroquine or methotrexate.

In some embodiments, the HIF-1 inhibitor is administered in combination a topical medication such as a cream, lotion or ointments that can reduce inflammation on the skin. Types of topical medications include corticosteroid creams and tacrolimus ointment (Protopic®).

In some embodiments, the HIF-1 inhibitor is administered in combination with an infusion treatment such as Anifrolumab, which is a monoclonal antibody medication used to treat certain autoimmune diseases.

### III. Compositions

Compositions for use in the disclosed methods are also provided. Such compositions are typically one or more HIF-1 inhibitors, or a formulation or pharmaceutical compositions including an effective amount thereof.

### A. HIF-1 Inhibitors

The results in the experiments below show that HIF-1 inhibitors can be useful for treating autoimmune skin diseases such as cutaneous lupus. Any suitable HIF-1 inhibitor can be used. The HIF-1 inhibitor can be, for example, a small molecule, a functional nucleic acid, an inhibitor protein, etc. HIF-1 inhibitors are known in the art, and non-limiting examples are provided below.

### 1. Exemplary Small Molecule HIF-1 Inhibitors

In some embodiments, the HIF-1 inhibitor is a small molecule.

Exemplary small molecule HIF inhibitors that can be used in the disclosed compositions and methods include, but are not limited to, those that are listed in Table 1:

TABLE 1
Published HIF Inhibitor Compounds

| | Method of | Mechanism of HIF | | |
|---|---|---|---|---|
| Name | identification | Inhibition | Biological Activities | Reference |
| Chemotin | HTS-p300-HIF | p300-HIF | Tumor suppression | (Kung et al., 2004) |
| | interaction | interaction | | |
| Topotecan | HTS- HRE-glioma | HIF translation | VEGF decrease | (Rapisarda et al., 2002) |
| | cell line | | | |
| 103D5R | HTS-HRE-glioma | HIF translation | Decreases HIF tested | (Tan et al., 2005) |
| | cell line | | target genes | |
| YC-1 | Direct testing | HIF protein levels | VEGF decrease/tumor | (Yeo et al., 2003 |
| | | | suppression | |
| GL331 | Direct testing | Protein levels | Inhibits HUVEC | (Chang et al., 2003) |
| | | | proliferation | |
| Geldanamycin | Direct testing | HSP90 inhibitor- | Tumor suppression | (Mabjeesh et al., 2002) |
| | | HIF half life | | |
| 2-ME2 | Direct testing | Post-transcriptional | Inhibits HUVEC | (Mabjeesh et al., 2003) |
| | | | proliferation | |
| Bisphenol | Direct testing | HIF degradation | No report | (Kubo et al., 2004) |
| Berberine | Direct testing | HIF degradation | No report | (Lin et al., 2004) |
| PX-478 | Thioredoxin | Unknown | Tumor suppression | (Welsh et al., 2004) |
| | inhibitor | | | |
| PX-12 | Thioredoxin | Unknown | Tumor suppression | (Welsh et al., 2004) |
| | inhibitor | | | |

Another HIF-1 inhibitor is the heteroaromatic acridine derivative acriflavine.

In some embodiments, the HIF-1 inhibitor is a nitrogen mustard compound. Nitrogen mustard compounds which are N-oxides and derivatives thereof are provided in U.S. Pat. No. 7,399,785, which is specifically incorporated by reference herein in its entirety. These compounds have the general formula set out below:

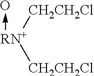

wherein R is an alkyl, aryl, aralkyl, or derivatives thereof such as CH₃OCH₂CH₂—, CH₃CH₂OCH₂CH₂—, C₆H₅OCH₂CH₂—, C₆H₅CH₂—, CH₃(CH₂)₃OCH₂CH₂Cl; or any one of the following:

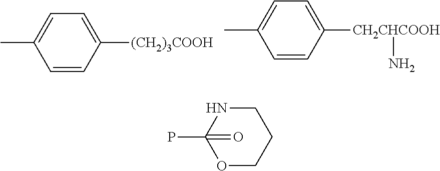

Also provided are salts of the above compounds. The salt would generally have the formulas set out above with a salt, wherein the salt and may be HCl, acetate, tosylate or picrate, and wherein R is as set out above.

Chlorambucil derivatives are described in U.S. Pat. No. 5,602,278, which is incorporated herein in its entirety. The '278 patent describes the use of chlorambucil and N-oxide derivates thereof in hypoxic environments, and more particularly chlorambucil in combination with hydralazine to create such reactive conditions. The '278 patent described in vitro and in vivo results the N-oxide derivative of chlorambucil (CHLN-O) and of the hydroxylamine derivative of chlorambucil (CHL-HD).

Thus, in a particular embodiment, the HIF-1 inhibitor is the chlorambucil derivative 4[p-(N−2-chloroethoxy N−2 chloroethylamino)phenyl]butanoic acid.

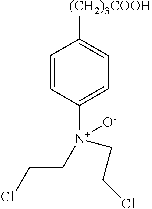

In a preferred embodiment, the HIF-1 inhibitor is PX-478 (S−2-amino-3-[4′-N,N,-bis(2-chloroethyl)amino]phenyl propionic acid N-oxide dihydrochloride) or melphalan N-oxide and derivatives thereof:

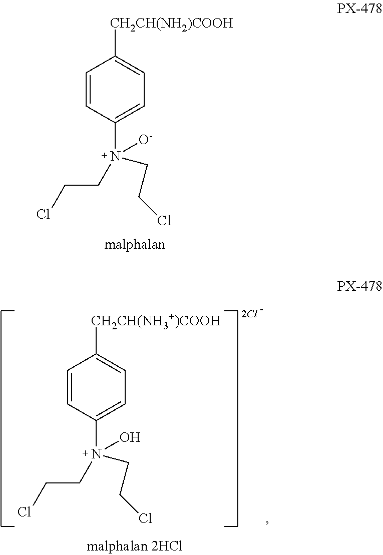

which significantly decreases the hypoxia-induced increase in HIF-1α protein but has little or no effect on HIF-1β. Oral PX-478 has been the subject of a clinical trial in patients with advanced solid tumors or lymphoma. See, e.g., ClinicalTrials.gov Identifier: NCT00522652.

Other HIF-1 inhibitors are disclosed in U.S. Pat. No. 10,881,656 the entire contents of which are specifically incorporated by reference herein its entirety, and can be of the structure shown in formula (I):

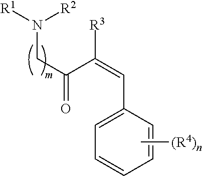

wherein:
R¹ and R² are each independently H, alkyl, alkenyl, alkynyl, or taken together with the N atom to which they are attached form a 5-8 membered heterocyclyl or heteroaryl, each of which can be optionally substituted;
R³ is H, alkyl, alkenyl, or alkynyl, each of which can be optionally substituted;
R⁴ is independently for each occurrence halogen, alkyl, alkenyl, alkynyl, alkoxy, CF₃, NO₂, N(R⁵)₂, CN, OH, SRS, SO₂R⁵, or two R⁴ taken together form an optionally substituted 3,4-methylenedioxy, each of which can be optionally substituted;
R⁵ is independently for each occurrence H, alkyl, alkenyl, or alkynyl, ach of which can be optionally substituted;
m is 1, 2, 3, 4 or 5;
n is 0, 1, 2, or 3; and
pharmaceutically acceptable salts thereof.

Although, the double bond is shown in the cis configuration, both the cis and trans double bond are amenable.

In some embodiments, at least one of R¹ and R² is C₁-C₆ alkyl. In some further embodiments of this, both of R¹ and R² are C₁-C₆ alkyl. When both of R¹ and R² are C₁-C₆ alkyl, they can be the same or different alkyl, any combination of the two alkyl groups being suitable. In some embodiments, both of R¹ and R² are methyl or ethyl.

In some other embodiments, at least one of R¹ and R² is H. In some further embodiments of this, both of R¹ and R² are H.

In yet some other embodiments, R¹ and R² taken together with the N atom they are attached to form a 5-8 membered heterocyclyl, e.g., morphilino, piperdino, pyrrolidino or piperaznio. Preferably heterocyclyl is morphilino.

When R³ is optionally substituted C₁-C₆ alkyl, R³ is selected from the group consisting of methyl, ethyl, propyl, isopropyl, n-butyl, isobutyl, and t-butyl. In some embodiments, R³ is methyl.

In some embodiments, m is 2.

In some embodiments, n is 0 or 1.

In general, on phenyl ring to which R⁴ can be attached, attachment of R⁴ at the 3- or 4-position is preferred. When n is 2, attachment of R⁴ at the 3- and 4-positions is preferred. When m is 2 or more, all of the R⁴ can all be the same, all different or a combination thereof.

In some embodiments, R⁴ is halogen, OH, optionally substituted C₁-C₆ alkyl or optionally substituted C₁-C₆ alkoxy.

Some exemplary compounds of formula (I) include, 5-(4-morpholinyl)-1-phenyl-1-penten-3-one; 5-(4-morpholinyl)-1-(3,4-dichlorophenyl)-1-penten-3-one; 5-(4-morpholinyl)-1-(3-trifluoromethylphenyl)-1-penten-3-one; 5-(4-morpholinyl)-1-(2,6-dichlorophenyl)-1-penten-3-one; 5-(4-morpholinyl)-1-(1,3-benzodioxyl-5-yl)-1-penten-3-one; 5-(4-morpholinyl)-1-(4-(methylthio)phenyl)-1-penten-3-one; 5-(4-morpholinyl)-1-(4-(methylsulfonyl)phenyl)-1-penten-3-one; 5-(4-morpholinyl)-1-(4-chlorophenyl)-1-penten-3-one; 5-(4-morpholinyl)-1-(4-cyanophenyl)-1-penten-3-one; 5-methylamino-1-phenyl-1-penten-3-one; 5-dimethylamino-1-(2-ethylphenyl)-1-penten-3-one; 5-(1-piperidino)-1-(2,4-dipropylphenyl)-1-penten-3-one; 5-(1-piperidino)-1-(2,4,6-trimethylphenyl)-1-penten-3-one; 5-(1-pyrrolidino)-1-(4-n-butoxyphenyl)-1-penten-3-one; 5-(1-piperazino)-1-(4-aininophenyl)-1-penten-3-one; 5-(4-ethylpiperazino-1-yl)-1-(4-nitrophenyl)-1-penten-3-one; 5-amino-1-(3-dim ethylaminophenyl)-1-penten-3-one; 5-(4-morpholinyl)-1-(4-hydroxyphenyl)-1-penten-3-one; and 5-(4-morpholinyl)-1-(2-methyl-4-chlorophenyl)-1-penten-3-one.

(In some embodiments, the compound of formula (I) is 5-dimethylamino)-2-methyl-1-phenyl-1-penten-3-one (COMPOUND 40).

In some embodiments, HIF-1 inhibitor is of structure shown in formula (II):

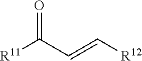

wherein:
R¹¹ is optionally substituted aryl, optionally substituted heteroaryl, optionally substituted cyclyl or optionally substituted heterocyclyl;
R¹² is optionally substituted aryl or optionally substituted heteroaryl; and pharmaceutically acceptable salts thereof.

Although, the double bond is shown in the trans configuration, both the cis and trans double bond are amenable to the invention.

R¹¹ can be substituted with a substituent selected from the group consisting of halogen, alkyl, alkenyl, alkynyl, alkoxy, aryl, heteroaryl, cyclyl, heterocyclyl, carbonyl (═O), CF₃, NO₂, N(R¹³)₂, CN, OH, SR¹³, SO₂R¹³, and combinations thereof, where each of alkyl, alkenyl, alkynyl, heterocyclyl, heteroaryl, acylcyl or heterocyclyl can be optionally substituted, and wherein R¹³ is independently for each occurrence H, alkyl, alkenyl or alkynyl. In some embodiments, R¹¹ is substituted with 1, 2, 3, or 4 substituents. In some embodiments, R¹¹ is substituted with OH, alkyl and carbonyl.

In some embodiments, R¹¹ is an optionally substituted heterocyclyl. In some embodiments, R¹¹ is selected from the group consisting of pyranyl, piperazinyl, pyrrolidinyl, dioxanyl, morpholinyl, tetrahydrofuranyl, each of which can be optionally substituted with 1 to 4 substituents. In some embodiments, R¹¹ is pyrane, e.g., substituted pyrane. In some embodiments, R¹¹ is a pyrane, wherein the pyrane is substituted with three substituents. In one embodiment, R¹¹ is

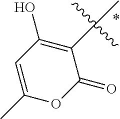 [SMILES: *c1c(O)cc(C)oc1=O]

R² can be substituted with a substituent selected from the group consisting of halogen, alkyl, alkenyl, alkynyl, alkoxy, aryl, heteroaryl, cyclyl, heterocyclyl, CF₃, NO₂, N(R¹⁴)₂, CN, OH, SR¹⁴, SO₂R¹⁴, and combinations thereof, where each of alkyl, alkenyl, alkynyl, heterocyclyl, heteroaryl, acylcyl or heterocyclyl can be optionally substituted, and wherein R¹⁴ is independently for each occurrence H, alkyl, alkenyl or alkynyl, each of which may be optionally substituted.

In some embodiments, R¹² is substituted with 1, 2, 3, or 4 substituents. In some embodiments, R¹² is substituted with OH and alkoxy.

In some embodiments, R¹² is an optionally substituted aryl. In some embodiments, R¹² is selected from the group consisting of phenyl, naphthyl, anthracenyl, azulenyl, fluorenyl, indanyl, indenyl, and tetrahydronaphthyl, each of which can be optionally substituted with 1 to 4 substituents.

In some embodiments, R¹² is

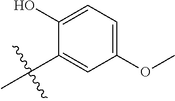 [SMILES: *c1cc(OC)ccc1O]

In some embodiments, the compound of formula (II) is 4-hydroxy-3-(3-(2-hydroxy-5-methoxyphenyl)acryloyl)-6-methyl-2H-pyran-2-one (COMPOUND 41).

Compounds of formula (I) and (II) can be synthesized by aldol-condensation of appropriate benzaldehydes and ketones. Synthesis of some exemplary compounds of formula (I) is described, for example in U.S. Pat. No. 4,400,380, the contents of which are specifically incorporated herein by reference in its entirety.

In some embodiments, the HIF-1 inhibitor is of structure shown in formula (III):

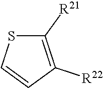

wherein:
R²¹ is halogen, alkyl, alkenyl, alkynyl, alkoxy, aryl, heteroaryl, cyclyl, heterocyclyl, CF₃, NO₂, N(R²³)₂, CN, OH, SR²³, SO₂R²³, or C(O)R²⁴, each of which can be optionally substituted;
R²² is alkoxy, halogen, alkyl, alkenyl, alkynyl, alkoxy, aryl, heteroaryl, cyclyl, heterocyclyl, CF₃, NO₂, N(R²⁵)₂, CN, OH, SR²³, or SO₂R²³, each of which can be optionally substituted;
R²³ is independently for each occurrence H, alkyl, alkenyl or alkynyl, each of which can be optionally substituted;
R²⁴ is alkyl, alkenyl, alkynyl, alkoxy, aryl, heteroaryl, cyclyl or heterocyclyl, each of which can be optionally substituted;
R²⁵ is independently for each occurrence alkyl, alkenyl, alkynyl, N(R²³)₂ or N=R²⁶, each of which can be optionally substituted;
R²⁶ is independently for each occurrence H, alkyl, alkenyl or alkynyl, each of which can be optionally substituted; and pharmaceutically acceptable salts thereof.

In some embodiments, R²¹ is C(O)R²⁴.

In some embodiments, R²⁴ is an alkyl or alkoxy. In some embodiments, R²⁴ is an alkoxy, e.g., C₁-C₆ alkoxy.

In one embodiment, R²⁴ is methoxy.

In some embodiments, R²¹ is C(O)CH₃, C(O)CH₂CH₃, C(O)CH₂CH₂CH₃, C(O)CH(CH₂)₂ or C(O)C(CH₃)₃.

In some embodiments, R²² is N(R²⁵)₂.

In some embodiments, at least one of R²⁵ is not H.

In some embodiments, R²⁵ is N=R²⁶.

In some further embodiments, R²⁶ is a substituted C₁-C₆ alkyl.

In some embodiments, R²⁶ is a C₁-C₆ alkyl, and which alkyl is substituted with SO₂R²³.

In some embodiments, R²⁶ is a C₁-C₆ alkyl, and which alkyl is substituted with CN.

In some embodiments, R²⁶ is a C₁-C₆ alkyl, and which alkyl is substituted with both SO₂R²³ and CN.

In some embodiments, R²⁶ is C(SO₂R²³)CN.

In some embodiments, R²⁶ is C(SO₂CH₃)CN.

In some embodiments, R²⁵ is N═C(SO₂CH₃)CN.

In one embodiment, the compound of formula (III) is methyl 3-{2-[cyano(methylsulfonyl)methyl]hydrazine]thiophene-2-carboxylate (COMPOUND 76).

In some embodiments, HIF inhibitor is of structure shown in formula (IV):

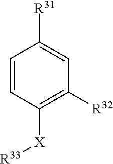

wherein:
X is S, O, or NH;
R³¹ and R³² are each independently halogen, alkyl, alkenyl, alkynyl, alkoxy, CF₃, NO₂, N(R³⁴)₂, CN, OH, SR³⁴, or SO₂R³⁴, each of which can be optionally substituted
R³³ is alkyl, alkenyl, alkynyl, aryl, heteroaryl, cyclyl, or heterocyclyl, each of which can be optionally substituted;
R³⁴ is independently for each occurrence H, alkyl, alkenyl, or alkynyl, each of which can be optionally substituted; and pharmaceutically acceptable salts thereof.

In some embodiments, R³¹ is an optionally substituted C₁-C₆ alkyl.

In some embodiments, R³¹ is an optionally substituted methyl or ethyl.

In some embodiments, R³¹ is an optionally substituted alkyne.

In some embodiments, R³¹ is an optionally substituted propyne.

In some embodiments, R³¹ is CN.

In some embodiments, R³² is NO₂ or N(R³⁴)₂.

In some embodiments, when R³² is N(R³⁴)₂, both R³⁴ are H.

In some other embodiments, when R³² is N(R³⁴)₂, at least one of the R³⁴ is not H.

In still some other embodiments, when R³² is N(R³⁴)₂, at least one of the R³⁴ is a C₁-C₆ alkyl.

In yet still some other embodiments, R³² is a dialkylamine.

In one preferred embodiment, R³¹ is NO₂.

In one embodiment, R³³ is an optionally substituted heterocyclyl. In some embodiments, R³³ is selected from the group consisting of pyranyl, piperazinyl, pyrrolidinyl, dioxanyl, morpholinyl, and tetrahydrofuranyl, each of which can be optionally substituted.

In some embodiments, R³³ is an optionally substituted thiopyrane. In some embodiments, R³³ is a thiopyrane substituted with at least substituent selected from the group consisting of halogen, alkyl, alkenyl, alkynyl, alkoxy, aryl, heteroaryl, cyclyl, heterocyclyl, carbonyl (═O), CF₃, NO₂, N(R³⁵)₂, CN, OH, SR³⁵, SO₂R³⁵, and combinations thereof, where each of alkyl, alkenyl, alkynyl, heterocyclyl, heteroaryl, acylcyl or heterocyclyl can be optionally substituted, and wherein R³⁵ is independently for each occurrence H, alkyl, alkenyl or alkynyl, each of which may be optionally substituted.

In some embodiments, R³³ is a mono, di, or tri substituted thiopyrane.

In some embodiments, R³³ is 2,3-dihydro-2,2-dimethylthiopyran-4-one.

In some embodiments, R³³ is

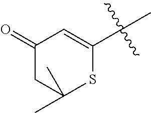

In one embodiment, the compound of formula (IV) is 4-[(2,2-dimethyl-4-oxo-3,4-dihydro-2H-thinn-6-yl)thio]-3-nitrobenzonitrile (COMPOUND 77).

In some embodiments, HIF inhibitor is selected from the group of compounds set forth in Table 2 and combinations thereof:

| Name | Full Chemical Name | Structure |
|---|---|---|
| 20 | 3-(2,5-diethoxyphenyl)-1-(2-thienyl)-2- propen-1-one | 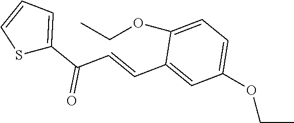 [SMILES: CCOc1ccc(OCC)c(/C=C/C(=O)c2cccs2)c1] |
| 37 | N2-(4-bromo-3-nitrobenzoyl) leucinamide | 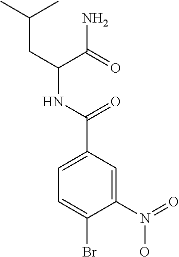 |
| 39 | 2-bromo-N-(2-methoxyphenyl) propenamide | 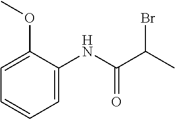 |
| 40 | 5-(dimethylamino)-2-methyl-1-phenyl- 1-penten-3-one hydrochloride | 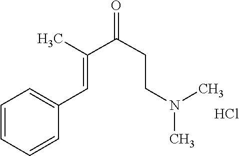 [SMILES: C/C(=C\c1ccccc1)C(=O)CCN(C)C ; Cl] |
| 41 | 4-hydroxy-3-[3-(2-hydroxy-5- meoxyphenyl)acryloyl]-6-methyl- 2H-pyran-2-one | 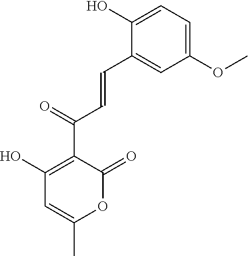 [SMILES: COc1ccc(O)c(/C=C/C(=O)c2c(O)cc(C)oc2=O)c1] |
| 42 | 6-(1,3-dioxo-1H- benzo[de]isoquinolin-2(3H)-yl)- N-hydroxyhexanamide | 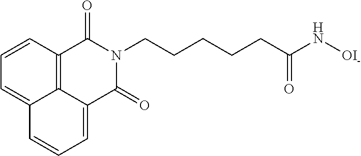 |
| 76 | methyl 3-{2- [cyano(methylsulfonyl)methylene] hydrazino}thiophene-2- carboxylate | 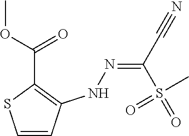 [SMILES: COC(=O)c1sccc1N/N=C(/C#N)S(C)(=O)=O] |
| 77 | 4-[(2,2-dimethyl-4-oxo-3,4- dihydro-2H-thiin-6-yl)thio]-3- nitrobenzonitrile | 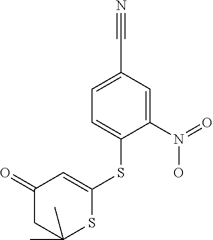 |

Hypoxia-inducible factor 1-alpha inhibitor-Hydroxylates HIF-1 alpha at ‘Asn-803’ in the C-terminal transactivation domain (CAD). Functions as an oxygen sensor and, under normoxic conditions, the hydroxylation prevents interaction of HIF-1 with transcriptional coactivators including Cbp/p300-interacting transactivator. Involved in transcriptional repression through interaction with HIF1A, VHL and histone deacetylases. Hydroxylates specific Asn residues within ankyrin repeat domains (ARD) of NFKB1, NFKBIA, NOTCH1, ASB4, PPP1R12A and several other ARD-containing proteins. Also hydroxylates Asp and His residues within ARDs of ANK1 and TNKS2, respectively. Negatively regulates NOTCH1 activity, accelerating myogenic differentiation. Positively regulates ASB4 activity, promoting vascular differentiation. 2. Exemplary Functional Nucleic Acids The HIF-1 inhibitor can be a functional nucleic acid. Functional nucleic acids are nucleic acid molecules that have a specific function, such as binding a target molecule or catalyzing a specific reaction. As discussed in more detail below, functional nucleic acid molecules can be divided into the following non-limiting categories: antisense molecules, siRNA, miRNA, aptamers, ribozymes, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Therefore the compositions can include one or more functional nucleic acids designed to reduce expression of a HIF-1 gene, or a gene product thereof. For example, the functional nucleic acid or polypeptide can be designed to target and reduce or inhibit expression or translation of a HIF-1 mRNA; or to reduce or inhibit expression, reduce activity, or increase degradation of a HIF-1 protein. In some embodiments, the composition includes a vector suitable for in vivo expression of the functional nucleic acid.

In some embodiments, a functional nucleic acid is designed to target a segment of a nucleic acid encoding the amino acid sequence of SEQ ID NO:1, or the complement thereof, or variants thereof having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a nucleic acid encoding the amino acid sequence of SEQ ID NO:1.

In some embodiments, a functional nucleic acid is designed to target a segment of the nucleic acid sequence of SEQ ID NO:2, or the complement thereof, or a genomic sequence corresponding therewith, or variants thereof having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:2.

In some embodiments, the function nucleic acid hybridizes to the nucleic acid of SEQ ID NO:2, or a complement thereof, for example, under stringent conditions. In some embodiments, the functional nucleic acid hybridizes to a nucleic acid sequence that encodes SEQ ID NO:2, or a complement thereof, for example, under stringent conditions.

a. Antisense

The functional nucleic acids can be antisense molecules. Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

b. Aptamers

The functional nucleic acids can be aptamers. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 1012 M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a K_{d} with the target molecule at least 10, 100, 1000, 10,000, or 100,000 fold lower than the K_{d} with a background binding molecule. It is preferred when doing the comparison for a molecule such as a polypeptide, that the background molecule be a different polypeptide.

c. Ribozymes

The functional nucleic acids can be ribozymes. Ribozymes are nucleic acid molecules that are capable of catalyzing a chemical reaction, either intramolecularly or intermolecularly. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

d. External Guide Sequences

The functional nucleic acids can be external guide sequences. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukarotic cells. Representative examples of how to make and use EGS molecules to facilitate cleavage of a variety of different target molecules are known in the art.

e. RNA Interference

In some embodiments, the functional nucleic acids induce gene silencing through RNA interference. Gene expression can also be effectively silenced in a highly specific manner through RNA interference (RNAi). This silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, et al. (2001) Genes Dev., 15:188-200; Bernstein, et al. (2001) Nature, 409:363-6; Hammond, et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, a siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs.

Sequence specific gene silencing can be achieved in mammalian cells using synthetic, short double-stranded RNAs that mimic the siRNAs produced by the enzyme dicer (Elbashir, et al. (2001) Nature, 411:494 498) (Ui-Tei, et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Texas), ChemGenes (Ashland, Massachusetts), Dharmacon (Lafayette, Colorado), Glen Research (Sterling, Virginia), MWB Biotech (Esbersberg, Germany), Proligo (Boulder, Colorado), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAse (shRNAs). Kits for the production of vectors having shRNA are available, such as, for example, Imgenex's GENESUPPRESSOR™ Construction Kits and Invitrogen's BLOCK-IT™ inducible RNAi plasmid and lentivirus vectors.

In some embodiment, the functional nucleic acid is siRNA, shRNA, miRNA. In some embodiments, the composition includes a vector expressing the functional nucleic acid. Methods of making and using vectors for in vivo expression of functional nucleic acids such as antisense oligonucleotides, siRNA, shRNA, miRNA, EGSs, ribozymes, and aptamers are known in the art.

### 3. Exemplary Inhibitor Proteins

In some embodiments, the HIF-1 inhibitor is an inhibitory polypeptide or protein. Exemplary protein inhibitors of HIF-1 are known in the art and include, e.g., a polypeptide having the amino acid sequence of UniPro Accession No. Q9NWT6·HIFIN_HUMAN: MAATAAEAVASGSGEPREEAGALGPAWDESQLRSYSFPTRPIPRLSQSDPRAEELIENE EPVVLTDTNLVYPALKWDLEYLQENIGNGDFSVYSASTHKFLYYDEKKMANFQNFKPRS NREEMKFHEFVEKLQDIQQRGGEERLYLQQTLNDTVGRKIVMDFLGFNWNWINKQQGKR GWGQLTSNLLLIGMEGNVTPAHYDEQQNFFAQIKGYKRCILFPPDQFECLYPYPVHHPC DRQSQVDFDNPDYERFPNFQNVVGYETVVGPGDVLYIPMYWWHHIESLLNGGITITVNF WYKGAPTPKRIEYPLKAHQKVAIMRNIEKMLGEALGNPQEVGPLLNTMIKGRYN (SEQ ID NO:5, UniPro Accession No. Q9NWT6·HIF1N_HUMAN), or a variant thereof with at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity thereto. See also, Hewitson, et al., J Biol Chem. 2002 Jul. 19; 277(29):26351-5. doi: 10.1074/jbc.C200273200. Epub 2002 May 31. PMID: 12042299.

In other embodiments, the HIF-1 inhibitor is cyclo-CLLFVY (SEQ ID NO:6), cyclo-CRLMVL (SEQ ID NO:7), or cyclo-CLLRMY (SEQ ID NO:8):

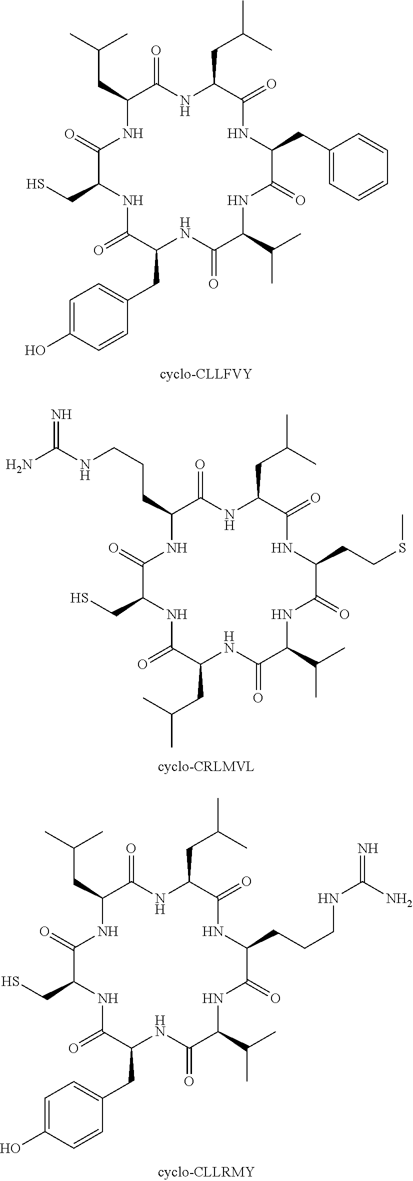 [SMILES: CC(C)C[C@@H]1NC(=O)[C@H](CS)NC(=O)[C@H](Cc2ccc(O)cc2)NC(=O)[C@H](C(C)C)NC(=O)[C@H](Cc2ccccc2)NC(=O)[C@H](CC(C)C)NC1=O ; CSCC[C@@H]1NC(=O)[C@H](CC(C)C)NC(=O)[C@H](CCCNC(=N)N)NC(=O)[C@H](CS)NC(=O)[C@H](CC(C)C)NC(=O)[C@H](C(C)C)NC1=O ; CC(C)C[C@@H]1NC(=O)[C@H](CS)NC(=O)[C@H](Cc2ccc(O)cc2)NC(=O)[C@H](C(C)C)NC(=O)[C@H](CCCNC(=N)N)NC(=O)[C@H](CC(C)C)NC1=O]

or a variant thereof with at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity thereto. See also Miranda, et al. Journal of the American Chemical Society 2013 135 (28), 10418-10425, DOI: 10.1021/ja402993u, which is specifically incorporated by reference herein in its entirety.

In some embodiments, the polypeptide HIF-1 inhibitor is delivered to the subject using a nucleic acid encoding the polypeptide, e.g., a vector, mRNA, etc. Thus, nucleic acids encoding polypeptide HIF-1 inhibitors are also provided as HIF-1 inhibitors, as are their use in the disclosed formulations and methods.

### B. Protein Transduction Domain

In some embodiments, the HIF-1 inhibitor, particularly, but not limited to, polypeptide or nucleic acid inhibitors, include a protein transduction domain (PTD) fused or otherwise conjugated thereto. Thus, the inhibitory proteins can be fusion proteins. As used herein, a “protein transduction domain” or PTD refers to a polypeptide, polynucleotide, carbohydrate, organic or inorganic compound that facilitates traversing a lipid bilayer, micelle, cell membrane, organelle membrane, or vesicle membrane. A PTD attached to another molecule facilitates the molecule traversing membranes, for example going from extracellular space to intracellular space, or cytosol to within an organelle.

In preferred embodiments, the protein transduction domain is a polypeptide. A protein transduction domain can be a polypeptide including positively charged amino acids. Thus, some embodiments include PTDs that are cationic or amphipathic. Protein transduction domains (PTD), also known as a cell penetrating peptides (CPP), are typically polypeptides including positively charged amino acids. PTDs are known in the art, and include but are not limited to small regions of proteins that are able to cross a cell membrane in a receptor-independent mechanism (Kabouridis, P., Trends in Biotechnology (11):498-503 (2003)). Although several PTDs have been documented, the two most commonly employed PTDs are derived from TAT (Frankel and Pabo, Cell, 55(6):1189-93(1988)) protein of HIV and Antennapedia transcription factor from Drosophila, whose PTD is known as Penetratin (Derossi et al., J Biol Chem., 269(14):10444-50 (1994)). Exemplary protein transduction domains include polypeptides with 11 Arginine residues, or positively charged polypeptides or polynucleotides having 8-15 residues, preferably 9-11 residues.

The Antennapedia homeodomain is 68 amino acid residues long and contains four alpha helices. Penetratin is an active domain of this protein which consists of a 16 amino acid sequence derived from the third helix of Antennapedia. TAT protein consists of 86 amino acids and is involved in the replication of HIV-1. The TAT PTD consists of an 11 amino acid sequence domain (residues 47 to 57; YGRKKRRQRR R (SEQ ID NO:16)) of the parent protein that appears to be critical for uptake. Additionally, the basic domain Tat(49-57) or RKKRRQRRR (SEQ ID NO:17) has been shown to be a PTD. In the current literature TAT has been favored for fusion to proteins of interest for cellular import. Several modifications to TAT, including substitutions of Glutatmine to Alanine, i.e., Q→A, have demonstrated an increase in cellular uptake anywhere from 90% (Wender et al., Proc Natl Acad Sci USA., 97(24):13003-8 (2000)) to up to 33 fold in mammalian cells. (Ho et al., Cancer Res., 61(2):474-7 (2001)).

The most efficient uptake of modified proteins was revealed by mutagenesis experiments of TAT-PTD, showing that an 11 arginine stretch was several orders of magnitude more efficient as an intercellular delivery vehicle. Therefore, PTDs can include a sequence of multiple arginine residues, referred to herein as poly-arginine or poly-ARG. In some embodiments the sequence of arginine residues is consecutive. In some embodiments the sequence of arginine residues is non-consecutive. A poly-ARG can include at least 7 arginine residues, more preferably at least 8 arginine residues, most preferably at least 11 arginine residues. In some embodiments, the poly-ARG includes between 7 and 15 arginine residues, more preferably between 8 and 15 arginine residues. In some embodiments the poly-ARG includes between 7 and 15, more preferably between 8 and 15 consecutive arginine residues. An example of a poly-ARG is RRRRRRR (SEQ ID NO:9). Additional exemplary PTDs include but are not limited to;

(SEQ ID NO: 10)
RRQRRTSKLM KR;
(SEQ ID NO: 11)
GWTLNSAGYL LGKINLKALA ALAKKIL;
(SEQ ID NO: 12)
WEAKLAKALA KALAKHLAKA LAKALKCEA;
and
(SEQ ID NO: 13)
RQIKIWFQNR RMKWKK

Without being bound by theory, it is believed that following an initial ionic cell-surface interaction, some polypeptides containing a protein transduction domain are rapidly internalized by cells via lipid raft-dependent macropinocytosis. For example, transduction of a TAT-fusion protein was found to be independent of interleukin-2 receptor/raft-, caveolar- and clathrin-mediated endocytosis and phagocytosis (Wadia, et al., Nature Medicine, 10:310-315 (2004), and Barka, et al., J. Histochem. Cytochem., 48(11):1453-60 (2000)). Therefore, in some embodiments the polynucleotide-binding polypeptide includes an endosomal escape sequence that enhances escape of the polypeptide-binding protein from macropinosomes. The some embodiments the endosomal escape sequence is part of, or consecutive with, the protein transduction domain. In some embodiments, the endosomal escape sequence is non-consecutive with the protein transduction domain. In some embodiments the endosomal escape sequence includes a portion of the hemagglutinin peptide from influenza (HA). One example of an endosomal escape sequence includes

(SEQ ID NO: 14)
GDIMGEWG NEIFGAIAGF LG.

In one embodiment a protein transduction domain including an endosomal escape sequence includes the amino acid sequence

(SEQ ID NO: 15)
RRRRRRRRRR RGEGDIMGEW GNEIFGAIAG FLGGE.

### C. Formulations

Formulations and pharmaceutical compositions including one or more HIF-1 inhibitors are also provided. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration.

The compositions are most typically administered systemically.

Drugs can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, desintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6^{th} Edition, Ansel et.al., (Media, PA: Williams and Wilkins, 1995) which provides information on carriers, materials, equipment and process for preparing tablets and capsules and delayed release dosage forms of tablets, capsules, and granules.

The compound can be administered to a subject with or without the aid of a delivery vehicle. Appropriate delivery vehicles for the compounds are known in the art and can be selected to suit the particular active agent. For example, in some embodiments, the active agent(s) is incorporated into or encapsulated by a nanoparticle, microparticle, liposome, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

### 1. Formulations for Parenteral Administration

Compounds and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### 2. Oral Immediate Release Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

3. Extended release dosage forms

The extended release formulations are generally prepared as diffusion or osmotic systems, for example, as described in “Remington—The science and practice of pharmacy” (20th ed., Lippincott Williams & Wilkins, Baltimore, MD, 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc. An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

### 4. Delayed Release Dosage Forms

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit®. L30D-55 and L100-55 (soluble at pH 5.5 and above), Eudragit®. L-100 (soluble at pH 6.0 and above), Eudragit®. S (soluble at pH 7.0 and above, as a result of a higher degree of esterification), and Eudragits®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### Methods of Manufacturing

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, PA: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

### 5. Formulations for Mucosal and Pulmonary Administration

Active agent(s) and compositions thereof can be formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one administration.

### 6. Topical and Transdermal Formulations

Transdermal formulations may also be prepared. These will typically be gels, ointments, lotions, sprays, or patches, all of which can be prepared using standard technology. Transdermal formulations can include penetration enhancers.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophillic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components.

Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Additional agents that can be added to the formulation include penetration enhancers. In some embodiments, the penetration enhancer increases the solubility of the drug, improves transdermal delivery of the drug across the skin, in particular across the stratum corneum, or a combination thereof. Some penetration enhancers cause dermal irritation, dermal toxicity and dermal allergies. However, the more commonly used ones include urea, (carbonyldiamide), imidurea, N, N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.). Chemical penetrations and methods of increasing transdermal drug delivery are described in Inayat, et al., Tropical Journal of Pharmaceutical Research, 8(2):173-179 (2009) and Fox, et al., Molecules, 16:10507-10540 (2011). In some embodiments, the penetration enhancer is, or includes, an alcohol such ethanol, or others disclosed herein or known in the art.

Delivery of drugs by the transdermal route has been known for many years.

Advantages of a transdermal drug delivery compared to other types of medication delivery such as oral, intravenous, intramuscular, etc., include avoidance of hepatic first pass metabolism, ability to discontinue administration by removal of the system, the ability to control drug delivery for a longer time than the usual gastrointestinal transit of oral dosage form, and the ability to modify the properties of the biological barrier to absorption.

Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir; matrix or monolithic devices are typically characterized by a falling drug flux with time, as the matrix layers closer to the skin are depleted of drug. Usually, reservoir patches include a porous membrane covering the reservoir of medication which can control release, while heat melting thin layers of medication embedded in the polymer matrix (e.g., the adhesive layer), can control release of drug from matrix or monolithic devices. Accordingly, the active agent can be released from a patch in a controlled fashion without necessarily being in a controlled release formulation.

Patches can include a liner which protects the patch during storage and is removed prior to use; drug or drug solution in direct contact with release liner; adhesive which serves to adhere the components of the patch together along with adhering the patch to the skin; one or more membranes, which can separate other layers, control the release of the drug from the reservoir and multi-layer patches, etc., and backing which protects the patch from the outer environment.

Common types of transdermal patches include, but are not limited to, single-layer drug-in-adhesive patches, wherein the adhesive layer contains the drug and serves to adhere the various layers of the patch together, along with the entire system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for control release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described in U.S. Pat. Nos. 6,461,644, 6,676,961, 5,985,311, and 5,948,433.

The disclosed compositions and method can be further understood through the following numbered paragraphs:

1. A method of treating an autoimmune skin disease or disorder in a subject comprising administering the subject an effective amount of a hypoxia-inducible factor-1 (HIF-1) inhibitor.

2. The method of paragraph 1, wherein the autoimmune disease is T cell mediated.

3. The method of paragraph 2, wherein the T cells are skin-infiltrating T cells.

4. The method of any one of paragraphs 1-3, wherein the HIF-1 inhibitor is administered in an effective amount to reduce the HIF-1 expression signature, reduce the cytotoxic activity, reduce expression of exhaustion marker(s), reduce Th17 phenotype, and/or reduce intracellular granzyme B in CD4+ and/or CD8+ T cells.

5. The method of any one of paragraphs 1-4, wherein the subject does not have lupus nephritis.

6. The method of any one of paragraphs 1-5, wherein the subject does not have cancer.

7. The method of any one of paragraphs 1-6, wherein the subject does not have an angiogenic disease or disorder.

8. The method of any one of paragraphs 1-7, wherein the subject does not have psoriasis, scleroderma, or pyogenic granulomas.

9. The method of any one of paragraphs 1-8, wherein the subject has cutaneous lupus, pemphigus, pemphigoid, epidermolysis bullosa acquisita vitiligo, lichen planus, lichen sclerosus, dermatomyositis, alopecia areata, or Sjögren's syndrome.

10. The method of any one of paragraphs 1-9, wherein the subject has cutaneous lupus selected from discoid cutaneous lupus, subacute cutaneous lupus, and acute cutaneous lupus.

11. The method of any one of paragraphs 1-9, wherein the subject does not have systemic lupus erythematosus (SLE).

12. The method of any one of paragraphs 1-11, wherein the HIF-1 inhibitor is a small molecule, functional nucleic acid, or inhibitory polypeptide or protein.

13. The method of any one of paragraphs 1-12, wherein the HIF-1 inhibitor is selected from PX-478, chemotin, topotecan, 103D5R, YC-1, GL331, geldanamycin, 2-ME2, bisphenol, berberine, and PX-12, or a pharmaceutically acceptable salt thereof.

14. The method of any one of paragraphs 1-12, wherein the HIF-1 inhibitor is a functional nucleic acid selected from antisense molecules, siRNA, miRNA, aptamers, ribozymes, RNAi, and external guide sequences.

15. The method of paragraph 13, wherein the functional nucleic acid targets a segment of a nucleic acid encoding the amino acid sequence of SEQ ID NO:1, or the complement thereof, or variants thereof having a nucleic acid sequence at least 65%, 70%, 75%, 80%, 85%, 90%, or 95% identical to a nucleic acid encoding the amino acid sequence of SEQ ID NO:1.

16. The method of paragraph 15, wherein the functional nucleic acid targets a segment of the nucleic acid sequence of SEQ ID NO:2, or the complement thereof, or a genomic sequence corresponding therewith, or variants thereof having a nucleic acid sequence at least 65%, 70%, 75%, 80%, 85%, 90%, or 95% identical to SEQ ID NO:2.

17. The method of any one of paragraphs 1-12, wherein the HIF-1 inhibitor is an inhibitor polypeptide comprising the amino acid sequence of any one of SEQ ID NOS:5-8, or variant thereof having a nucleic acid sequence at least 65%, 70%, 75%, 80%, 85%, 90%, or 95% identical to SEQ ID NO:5-8, or a nucleic acid encoding the same, optionally wherein the HIF-1 inhibitor has a structure selected from:

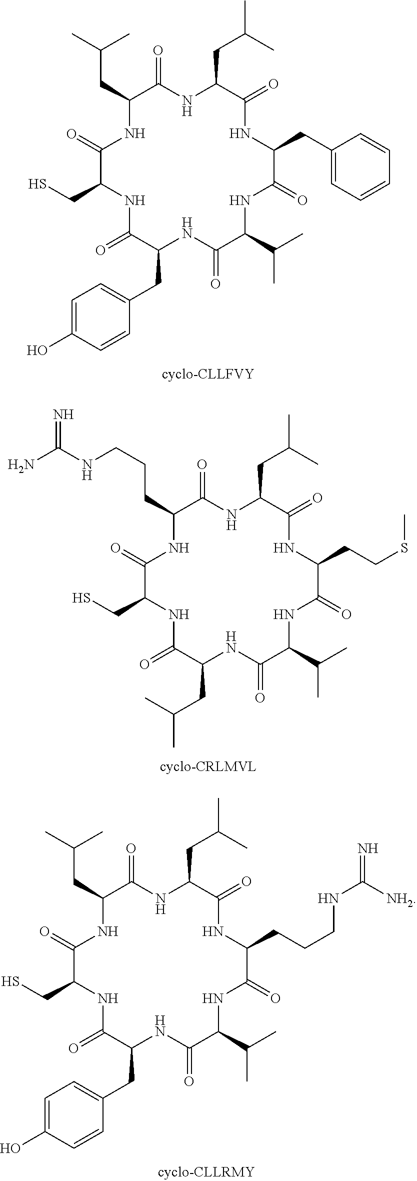 [SMILES: CC(C)C[C@@H]1NC(=O)[C@H](CS)NC(=O)[C@H](Cc2ccc(O)cc2)NC(=O)[C@H](C(C)C)NC(=O)[C@H](Cc2ccccc2)NC(=O)[C@H](CC(C)C)NC1=O ; CSCC[C@@H]1NC(=O)[C@H](CC(C)C)NC(=O)[C@H](CCCNC(=N)N)NC(=O)[C@H](CS)NC(=O)[C@H](CC(C)C)NC(=O)[C@H](C(C)C)NC1=O ; CC(C)C[C@@H]1NC(=O)[C@H](CS)NC(=O)[C@H](Cc2ccc(O)cc2)NC(=O)[C@H](C(C)C)NC(=O)[C@H](CCCNC(=N)N)NC(=O)[C@H](CC(C)C)NC1=O]

18. The method of any one of paragraphs 1-17, wherein the HIF-1 inhibitor further comprises a protein transduction domain.

19. The method of any one of paragraphs 1-18, wherein the HIF-1 inhibitor is a pharmaceutical composition further comprising a carrier.

20. The method of any one of paragraphs 1-19, wherein the HIF-1 inhibitor is administered systemically.

21. The method of any one of paragraphs 1-20, wherein the HIF-1 inhibitor is administered locally to skin effected by the autoimmune disease.

22. The method of paragraphs 21, wherein the HIF-1 inhibitor is administered by topical administration, injection, or intralesional administration.

23. The method of paragraphs 22, wherein the HIF-1 inhibitor is administered by topical administration in a pharmaceutical composition suitable for topical administration.

24. The method of paragraphs 23, wherein the pharmaceutical composition comprises a penetration enhancer.

25. The pharmaceutical composition of any one of paragraphs 19-24.

26. A pharmaceutical composition comprising a HIF-1 inhibitor for use in the method of any one of paragraphs 1-24.

27. The pharmaceutical composition of paragraphs 25 or 26 for topical administration.

28. The pharmaceutical composition of any one of paragraphs 25-27 comprising a penetration enhancer.

29. The pharmaceutical composition of any one of paragraphs 25-28 wherein the composition is a lotion or cream.

30. A patch comprising the pharmaceutical composition of any one of paragraphs 25-29.

### Examples

### Materials and Methods

### Mice

All mice were housed in the pathogen-free facility in the Yale Animal Resources Center (Yale University, New Haven, CT), and the experimental protocols were approved by the Yale Institutional Animal Care & Use Committee (IACUC) #2022-07801. MRL/MpJ-Faslpr/J (MRL/lpr) mice were purchased from the Jackson Laboratory.

### Human Skin Samples

Samples of DLE and control healthy skin from excisions from archived FFPE tissue were obtained from Yale Dermatopathology biorepository. Use of archived human FFPE tissue was approved by Yale University Human Investigative Committee #15010105235.

### Isolation of Murine Skin-Infiltrating T Cells

Fresh murine skin samples of diseased skin were collected from the interscapular region of 20- to 22-week-old MRL/lpr mice immediately after sacrifice. Skin was scraped with a razor blade on ice to remove subcutaneous fat and minced as previously described (41). Minced skin was digested in RPMI (Corning) containing 500 μg/ml Liberase TL (Millipore Sigma, #5401020001) and 104 U/mL DNase I (MP Biomedicals) at 37° C. on an orbital incubator for 70 minutes. Digested skin was mashed through a 70 μm filter (Falcon), washed with RPMI, filtered again through a 70 μm filter, and either stained immediately for flow cytometry/cell sorting or plated for in vitro stimulation experiments, followed by staining.

In vivo Pimonidazole Labelling

To detect regions of hypoxia in vivo, mice were injected with 80 mg/kg of pimonidazole (Hypoxyprobe™, HP-200 mg) 1.5 hours prior to sacrifice as previously described (42). Mice designated for flow cytometric analysis were injected with anti-CD45.1-PE five minutes prior to sacrifice to label circulating cells. Fresh dorsal skin and spleen samples were collected for T cell isolation, flow cytometry, immunofluorescence, and RNA analysis. Lesional and non-lesional interscapular skin was harvested to generate samples of approximately 1-2 cm² and were processed for flow cytometry as above or immediately fixed with periodate-lysine-paraformaldehyde (PLP) and subsequently embedded in OCT for immunofluorescence studies or immediately fixed with 10% normal buffered formalin with subsequent embedding into paraffin for RNAscope® experiments.

### Pharmacologic HIF-1 Blockade

Mice with no to early skin lesions were paired and treated with either PX-478 (Medchemexpress, HY-10231) by oral gavage at the dose of 5 mg/kg every 2 days for 4 weeks to inhibit HIF-1 in vivo, while control mice received an equivalent amount of PBS (4 ml/kg). Treatment was started at 16 weeks of age and ended at the age of 20 weeks. For single-cell RNA sequencing experiments, 20-week-old MRL/lpr mice were orally gavaged at the dose of 30 mg/kg daily for 5 days to block HIF-1 in vivo while control mice were gavaged with the same volume of PBS.

### Mouse Skin Assessment and Histopathologic Scoring

Skin was assessed at 20 weeks of age (end of treatment). Photographs were taken and were stripped of identifiers such that gross skin lesion score could be assessed in a blinded manner. Gross skin lesions were then graded by two blinded scorers (A.J.L and M.D.V.) on a scale of 0 to 3:0—none; 1—mild (snout and ears); 2—moderate (snout, ears, and interscapular; <1 cm per lesion); 3—severe (snout, ears, and interscapular; >1 cm per lesion or severe clinical assessment) (43). The majority of mice received identical scores from both blinded scorers, and all scores were within 1 point; average dermatitis score is reported for any mouse that received non-identical scores.

At 20 weeks of age (end of treatment), mice were sacrificed and shaved interscapular skin samples were immediately collected, fixed in 10% buffered formalin, and paraffin embedded. Hematoxylin and eosin-stained sections were scored in a blinded manner by one observer (J.M.) according to a semi quantitative scale (0-2) for acanthosis, hyperkeratosis, interface (liquefaction), and inflammation (44), with total histopathologic disease score per mouse reported as the sum of these sub scores.

Flow Cytometry and Cell Sorting and In vitro Stimulation

Skin tissue was processed as above. Spleen tissues were homogenized by crushing with the head of a 1 mL syringe in a petri dish followed by straining through a m nylon filter. ACK buffer was used for red cell lysis and remaining cells were counted. Antibodies used for flow cytometry staining included anti-mouse CD3 (Clone 17A2, BioLegend, 100228) anti-mouse TCRP (Clone H57-597, BD, 553170), anti-mouse CD4 (Clone RM4-5, Biolegend, 100548), anti-mouse CD8a (Clone 53-6.7, Biolegend, 100759), anti-human/mouse CD44 (Clone IM7, eBioscience, 47-0441-82), anti-mouse CD45 (Clone 30-F11, BD, 552848), anti-mouse CD45.1 (Clone A20, ThermoFisher, 12-0453-82), anti-mouse CD45R (Clone RA3-6B2, BD, 562290), anti-mouse CCR6 (Clone 140706, BD Horizon, 564736) anti-pimonidazole FITC-Mab (Clone 4.3.11.3, Hypoxyprobe, HP6-200), LIVE/DEAD™ Fixable Aqua Dead Cell Stain Kit (Invitrogen, L34966), anti-HIF-1α (Clone Hlalpha67, Novus Biologicals, NB-100-105AF647), anti-RORgt (Clone B2D, eBioscience, 12-6981-82), anti-T-bet (Clone eBio4B10, eBioscience, 12-5825-82), anti-granzyme B (Clone GB11, Invitrogen, GRB05), anti-IFN-7 (Clone XMG1.2, eBioscience, 50-7311-82), and anti-IL17a (Clone TC11-18H10.1, Biolegend, 506910). All stained samples were analyzed using an LSRII Multilaser Cytometer at Flow Cytometry Facility, Yale University, and were analyzed by FlowJo 10.7.1. Cytokine staining was performed after in vitro stimulation during which freshly isolated skin and spleen cells were plated at a density of 1×10\hat{ }6 live cells/ml and stimulated with RPMI complete media containing 50 ng/mL phorbol 12-myristate 13-acetate and 1 μg/mL ionomycin with Brefeldin A for 4 hours at 37° C. After surface marker staining, intracellular cytokine staining was performed with BD Cytofix/Cytoperm™ and perm/wash buffer.

### RNA Sequencing

Skin-infiltrating and splenic CD4+ and CD8+ T cells were isolated and sorted from lesional skin and spleens of 20-week-old MRL/lpr mice as above, and mRNA isolated (RNeasy Plus Micro Kit, Qiagen, 74034). SMART-Seq v4 Ultra Low Input RNA Kit for Sequencing (TaKaRa, 634893) was used to construct the sequencing library. Samples were sequenced on an Illumina HiSeq 2000 with 100-bp paired ends (Yale Center for Genome Analysis Core facility). Sequence alignment to mouse genome MM9 was done by using STAR 2.5.3 and TopHat 2.1.0 on the Partek Flow® platform. The differentially regulated genes were analyzed by DEseq2 (45).

For single-cell RNA sequencing with simultaneous surface protein detection of CD4, CD8a, and CD45RB, lesional skin and spleens of 20-week-old MRL/lpr mice were harvested one day after treatment with either PBS or PX-478 at the dose of 30 mg/kg daily for five days. After preparation and staining of single-cell suspensions from skin as above, live CD45+ and CD45− single cells were sorted and admixed at a 50:50 ratio. These cells and whole splenocytes prepared as above were then stained for surface protein (Feature Barcoding Technology) per BioLegend protocol with 0.5 μg of each oligonucleotide-tagged TotalSeqTM-C antibody: anti-mouse CD4 (BioLegend, Cat #100571), anti-mouse CD8a (BioLegend, Cat #100785), and anti-mouse CD45RB (BioLegend, Cat #103321). Cells were then submitted immediately for 10× single-cell RNA sequencing at Yale Center for Genome Analysis. The libraries were prepared by Yale Center for Genome Analysis and were sequenced by Novaseq 6000. The sequenced files were aligned using standard 10× Pipeline, CellRanger version 5.0.1. High-quality cells, defined as at least 200 detected genes, with percentage of mitochondrial genes per cell lower than 10%, were used for downstream analysis, and cell clustering was done with Seurat version 4.1.1. Cluster identities were determined using the CIPR (cluster identity predictor) package in R (46), and confirmed by manual review of cell-type specific transcripts by cluster. Single-cell gene set enrichment analysis was done by escape version 1.6.0 (47), referencing the following gene sets: Th17 cell differentiation from KEGG 2021 (48), T Cell Mediated Cytotoxicity from Jax Mouse Genome Informatics Gene Ontology Project (GO:0001913) (49), and HIF1α regulated genes derived from GSE35111 (20).

### Gene Set Enrichment Analysis

Gene set enrichment analyses (GSEA) were performed using GSEA software (v3.0) maintained by The Broad Institute (50).

### Immunofluorescence Microscopy

For murine skin immunofluorescence samples, immediately after sacrifice, mouse skin tissues were fixed with periodate-lysine-paraformaldehyde fixative (PLP) overnight. Samples were then dehydrated with 30% sucrose, embedded in optimal cutting temperature compound (Tissue-Tek), and stored at −80° C. Sections were cut at 7 m and stained with the following antibodies: anti-mouse CD4 (Clone RM4-5, Biolegend 100547, 1:100), anti-mouse CD8a (Clone 53-6.7, Biolegend, 100747, 1:100), anti-pimonidazole mouse FITC- or DyLight 549-Mab (Clone 4.3.11.3, Hypoxyprobe, HP6-200 or HP7-200, 1:100), rabbit polyclonal anti-HIF-1α (GeneTex, GTX127309, 5 μg/ml) or rabbit polyclonal isotype control (GeneTex, GTX35035, 5 μg/ml), and AlexaFluor 647-conjugated goat anti-rabbit secondary antibody (ThermoFisher Cat #A-21244, 1:1000). Confocal microscopy was done by Leica SP8 or SP5 laser scanning confocal microscope at the Cell Imaging Core, Yale Stem Cell Center.

### RNAscope In Situ Hybridization

The RNAscope 2.5HD RED assay kit (Advanced Cell Diagnostics) was used to perform in-situ hybridization for RNA detection in human DLE and healthy skin samples as well as MRL/lpr skin samples collected from PX-478 treatment cohorts. Slides were prepared and hybridized with RNA probes according to the manufacturer's instructions for FFPE samples. RNA probes included human and mouse GZMB (Cat #445971, Cat #49019) and IL17A (Cat #310931, Cat #319571), positive control peptidyl-prolyl isomerase B (PPIB) housekeeping gene (Cat #313901, Cat #313911) and negative control DapB soil bacteria enzyme (Cat #310043). Amplification and signal detection steps were performed according to kit instructions. Slides were then counterstained using hematoxylin and bluing reagent (Scigen Cat #23-730-614) and cover slipped using Permount (FisherSci Cat #SP15-100).

Immunohistochemistry staining was performed on human DLE samples after RNA in situ hybridization using the RNAscope 2.5HD RED assay kit. A second round of antigen retrieval was performed in citrate buffer (ThermoFisher Cat #00500) for 30 minutes and a peroxidase block was applied using 3% hydrogen peroxide (JT Baker cat #JT-2186-01) for 30 minutes. Subsequent blocking and staining steps were executed according to the ImmPRESS HRP Reagent kit (Cat #MP-7401) using normal horse serum. Slides were stained with primary anti-human CD3e (Cell Signaling Technology Cat #85061, 1:150) at 4° C. overnight and HRP secondary for 1 hr at room temperature. Signal was developed with diaminobenzidine (DAB) substrate (Vector Laboratories #SK-4100) prior to counterstaining and coverslipping as above.

Fluorescent RNA in situ hybridization with simultaneous immunofluorescent (IF) protein detection (FISH-IF) in human DLE samples was performed according to the ACD integrated co-detection protocol using the RNAscope Multiplex Fluorescent v2 Assay kit (Advanced Cell Diagnostics). Slides were prepared according to manufacturer's instructions for FFPE samples using the RNA-Protein Co-detection Ancillary Kit (ACD Cat #323180). Slides were incubated overnight with anti-CD3e primary antibody (clone SP7, ThermoFisher, MA5-14524, 1:50) and processed according to manufacturer's instructions for integrated protein-RNA co-detection. RNA probes included human GZMB (Cat #445971), positive control PPIB (Cat #320861) and DapB soil bacteria (Cat #320871). Amplification and signal detection steps were performed according to kit instructions for channel 1 and stained using Opal 570 (Akoya, Cat #FP1488001KT, 1:1500). Slides were stained with AlexaFluor 647-conjugated goat anti-rabbit secondary antibody (ThermoFisher Cat #A-21244, 1:200), and counterstained using DAPI. Slides were coverslipped with Prolong Gold Antifade (ThermoFisher Cat #P36930) and imaged using the Leica SP8 Gated STED 3× super resolution microscope at the Yale Center for Cellular and Molecular Imaging.

### RNA In Situ Hybridization Quantification

Slides stained by RNAscope 2.5HD RED assay kit were imaged using SPOT image software on a Zeiss Axioskop 40 light microscope under 400× magnification. RNA staining was quantified using Qupath Bioimage analysis software version 0.2.3 with the ImageJ Fiji v.2.10 extension. Quantification was performed using the bright field image nucleus detection program StarDist (arXiv: 1806.03535) with the he_heavy_augment model (51). The threshold for positive staining was set at one dot per cell and each sample was manually checked for accurate classification of positive staining in a blinded fashion. The number of positive staining cells was expressed as a percentage of the total cells in each high-powered field.

Slides stained by RNAscope Multiplex Fluorescence v2 Assay kit were quantified using HALO image analysis software (HALO 3.5.3577.214 and HALO AI 3.5.3577). Quantification was performed using the HALO FISH-IF algorithm (version 2.1.5) with the HALO Spatial Analysis module (version 3.5). T cells were identified by surface staining with CD3, and granzyme B-positive cells were identified with a threshold for positive staining set at four dots per cell. Samples were manually checked for accurate classification of positive staining. Spatial distribution of granzyme B-positive T cells was calculated by defining the dermo epidermal junction (DEJ) or border of the hair follicle and analyzing the dermal infiltrate for granzyme B-positive CD3+ cells as a function of distance from the defined border, in 50 micron “bins”, from 0-300 or 350 microns away from the DEJ or hair follicle, respectively. The number of granzyme B-positive staining CD3+ T cells was expressed as a percentage of the total CD3+ T cells in each of the 50 micron “bins”, and was plotted as percent of granzyme B-positive T cells as a function of distance (FIGS. 9A and 9B).

### Nanostring GeoMx Digital Spatial Profiling (DSP)

Samples of DLE and control skin from cyst excisions from archived FFPE tissue were obtained from Yale Dermatopathology biorepository. Sections of 5 μm were cut and placed on glass slide with each glass slide including three samples from the same disease or control state prior to shipping slides to Nanostring for GeoMx® DSP. The specific regions of interest (ROIs) for molecular profiling were then selected based on location of CD3-positive staining.

ROIs were profiled using the GeoMx® Digital Spatial Profiler (NanoString) (52). Slides were incubated with a multiplexed cocktail of RNA oligonucleotide probes with UV photocleavable indexing oligonucleotides (Cancer Transcriptome Atlas) and 4 fluorescent markers [Syto83 at 500 M for nuclei visualization; CD3-AF594 (Novus, C₃e/1308; 1:100); CD8-647 (Novus, SPM548; 1:200); and PanCK-AF488 (Novus, AE1/AE3; 1:500)]. For RNA analysis each ROI was geometric shape approximately 300 m in diameter with three CD3-rich ROIs selected per sample. ROIs were then exposed to UV illumination to cleave DNA oligos from the tissue. Cleaved oligos were collected through microcapillary aspiration and placed in a microwell plate. Oligos from each ROI were contained in separate wells. For Cancer Transcriptome Atlas, collected oligos were amplified using a forward primer and a reverse primer that serve as Illumina i5/i7 unique dual indexing sequences to index ROI identity. After purifying the PCR products with AMPure XP beads (Beckman Coulter), they were sequenced. Library purity and concentration were measured with DNA Bioanalyzer chip (Agilent). Reads after sequencing, were trimmed, merged, and aligned to retrieve the identity of probes. PCR duplicates and duplicate reads were removed, and the reads were converted to digital counts. The RNA sequencing saturation was sufficient and above 50%. After removing the outlier probes, the mean of the individual probe counts is considered as the reported count value. Using GeoMX software (NanoString), 75% upper quartile (Q3) of the counts per ROI were selected after removing genes with zero counts. The Q3 normalized counts were compared across ROI and disease subtypes using several approaches.

### Data Availability

The MRL/lpr bulk and single-cell RNA sequencing data are available in the Gene Expression Omnibus (GEO) database under accession number GSE229407. The human DLE Nanostring GeoMx Digital Spatial Profiling transcriptomics data are available on Mendeley Data (doi:10.17632/ck9f9rkdvw.2)

### Statistical Analysis

Data were analyzed using the appropriate indicated statistical test (e.g., Student's t-test, Mann-Whitney test, Spearman correlation, or Kruskal-Wallis test) with Prism 8 (GraphPad Software). The degree of significance was represented by the number of asterisks with respect to p value, *p<0.05, **p<0.01, *** p<0.001, **** p<0.0001.

### Results

Skin and kidney are among the most frequent targets of organ damage in systemic lupus erythematosus (SLE, lupus), and the pathological features include autoantibody deposition and subsequent T cell-mediated tissue injury (1, 2). The changes in environment experienced by T cells as they transition from systemic circulation to local injured tissue likely shape the phenotype and function of T cells upon infiltration into the organ and affect the extent of damage. Indeed, skin-infiltrating myeloid cells from lupus patients appear to experience such a transition upon migration into the skin, upregulating chemokines and interferon-responsive genes that may result in dendritic cell activation and promote local inflammation (3). Similarly, it has been demonstrated that oxygen tension deteriorates locally in lupus nephritis, and the subsequent upregulation of the transcription factor hypoxia inducible factor-1 (HIF-1) dictates the phenotype of kidney-infiltrating T cells and their promotion of tissue damage (4). Blocking HIF-1 by either genetic modification or pharmacological blockade dramatically reduced infiltrating T cell numbers and reversed kidney damage. However, whether HIF-1 plays a role in the development of tissue damage in other organ systems affected in SLE remains unknown.

Tissue damage in skin and kidney may share similar mechanisms; however, there are differences in the tissue environments between skin and kidney, and there is apparent stochasticity in development of damage to different organ systems within lupus patients. Here, to address this knowledge gap and determine whether HIF-1 regulates effector function of skin-infiltrating T cells to cause tissue damage in CLE, the phenotype and function of skin-infiltrating T cells were examined in lesional murine CLE skin as compared with their peripheral T cell counterparts, and the effect of HIF-1 inhibition on skin disease manifestations and skin-infiltrating T cell phenotype was determined. To confirm the applicability of the murine findings in the treatment of human disease, skin biopsies from patients with discoid lupus erythematosus (DLE), the most common subtype of CLE, were evaluated for HIF-1 expression and T cell phenotype as compared with healthy human skin.

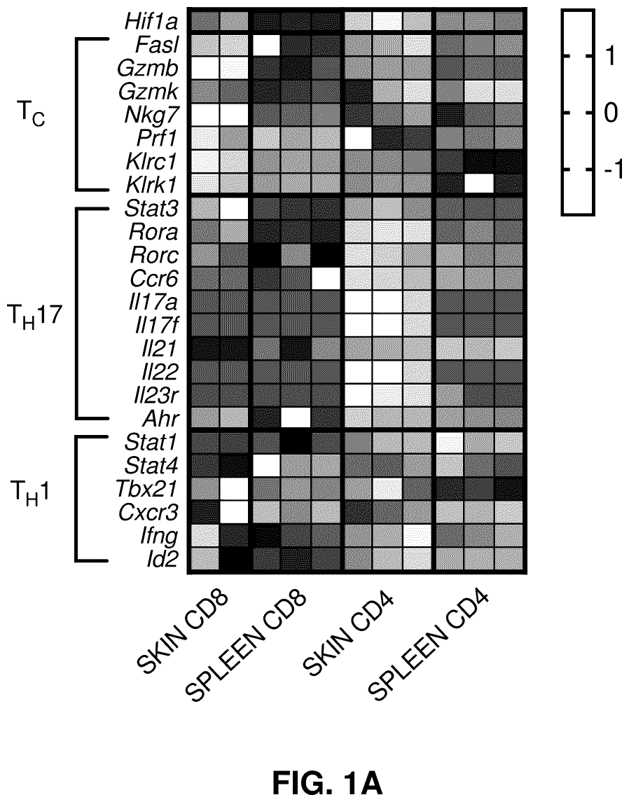

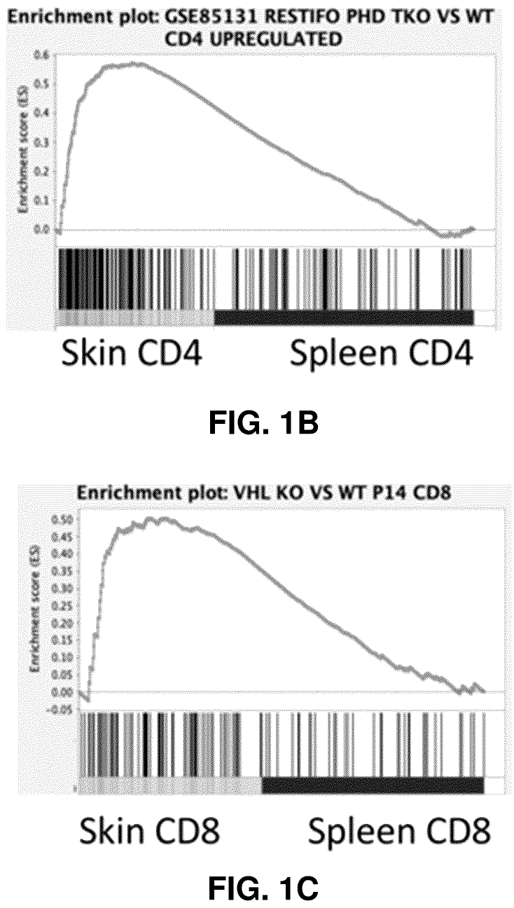

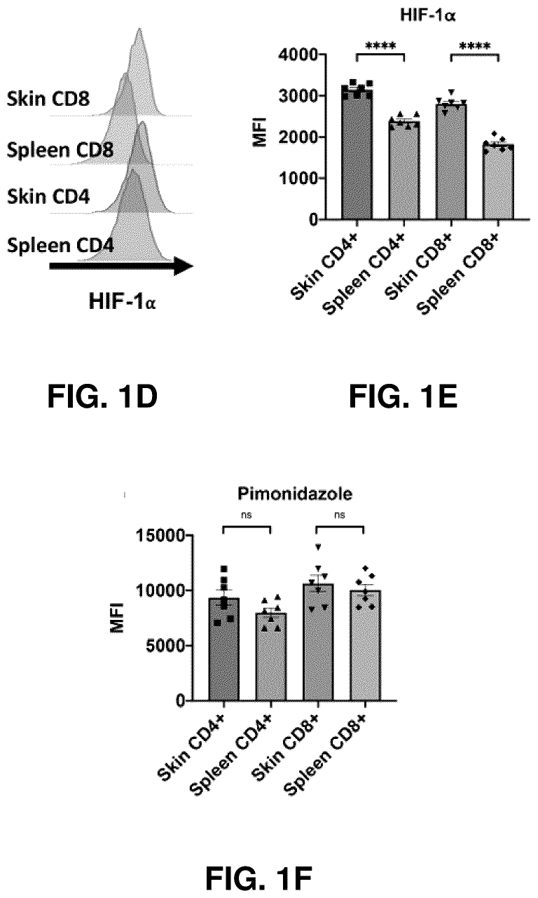

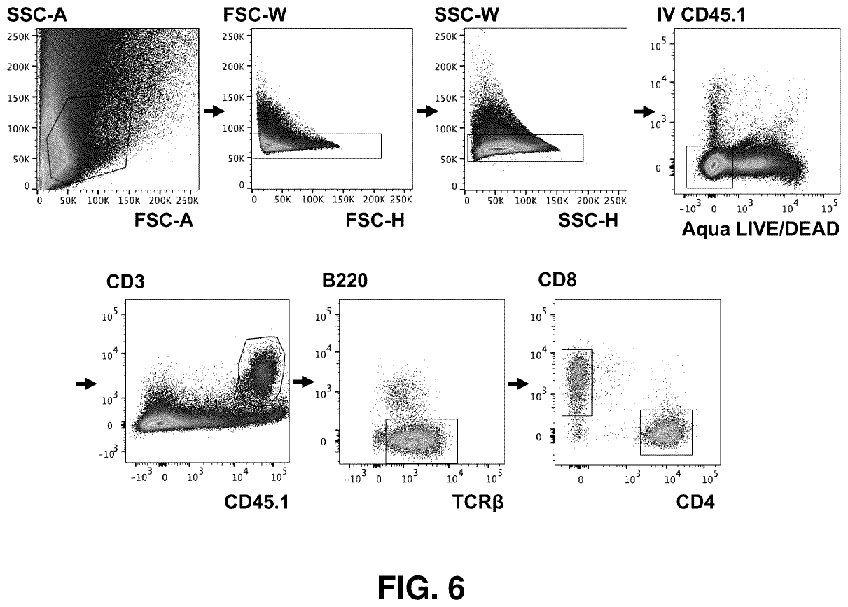

To examine phenotypes specific to skin-infiltrating T cells, CD4⁺ and CD8⁺ T cells from lesional dorsal skin of 20-week-old MRL/MpJ-Fas^{lpr}/J (MRL/lpr) lupus-prone mice were sorted, separating the tissue-resident cells from circulating cells (FIG. 6). CD4⁺ and CD8⁺ T cells from the spleens of these same mice were simultaneously sorted and bulk RNA sequencing was performed, comparing skin-infiltrating T cells to their splenic counterparts (FIG. 1A) to enable identification of transcriptional programs specifically upregulated in skin-infiltrating T cells from murine CLE skin.

Skin-infiltrating T cells show a dominant HIF-1 signature at the transcript and protein level.

As compared with splenocytes, skin infiltrating CD4⁺ and CD8⁺ T cells demonstrated significantly upregulated Hif1a transcript (FIG. 1A, top row). In addition to the upregulation of Hif1a transcript, gene set expression analysis (GSEA) of skin-infiltrating T cells as compared with splenic T cells revealed a dominant HIF-1 signature, as determined by comparison with previously-identified sets of genes that are upregulated in the setting of HIF-1 overexpression in CD4⁺ (17) or CD8⁺ T cells (9) (FIGS. 1B and 1C). Thus, in skin-infiltrating CD4⁺ or CD8⁺ T cells from murine CLE skin, observed was upregulation of both HIF1a transcripts and genes known to be activated by HIF-1 overexpression, as compared with splenic CD4⁺ or CD8⁺ T cells from the same animals.

The upregulation of HIF-1 as identified by RNA sequencing was confirmed by flow cytometry. HIF-1α protein was more highly expressed in both CD4⁺ and CD8⁺ skin-infiltrating T cells as compared to their counterparts taken simultaneously from spleens of MRL/lpr mice (FIGS. 1D and 1E). The presence of nuclear HIF-1α was confirmed by immunofluorescence staining in skin-infiltrating CD4⁺ and CD8⁺ T cells in lesional interscapular MRL/lpr skin (FIGS. 1G and 1H). To determine if upregulation of HIF-1α in skin-infiltrating T cells resulted from local hypoxia, pimonidazole (Hypoxyprobe™), which binds to thiol groups contained in proteins and peptides at oxygen tensions below 10 mmHg (18), identifying moderate to severely hypoxic conditions (14), was injected into mice before sacrifice. Unlike what was observed in renal-infiltrating T cells (4), the local microenvironment of skin-infiltrating T cells was not demonstrably hypoxic when evaluated by staining for pimonidazole, as the difference in pimonidazole staining between skin-infiltrating and splenic T cells was not significantly different (FIG. 1F). Despite identification of hypoxic areas in the dermis of diseased skin as identified by pimonidazole staining, a substantial amount of T cell infiltration was also seen in regions without increased pimonidazole staining, consistent with our flow cytometry findings (FIG. 1F). These data demonstrate upregulation of HIF-1α in T cells in murine CLE skin, which may be the result of non-hypoxic mechanisms rather than the direct response to hypoxia. However, it is also possible that diseased skin experiences decreased oxygen tension resulting in modest hypoxia (oxygen partial pressure of −19 mmHg) (14) that is not detectable using pimonidazole, which requires extremely low oxygen tension (<10 mmHg O₂) to form the stable thiol adducts that are detected (18).

Skin-infiltrating T cells demonstrate a cytotoxic and Th17 phenotype.

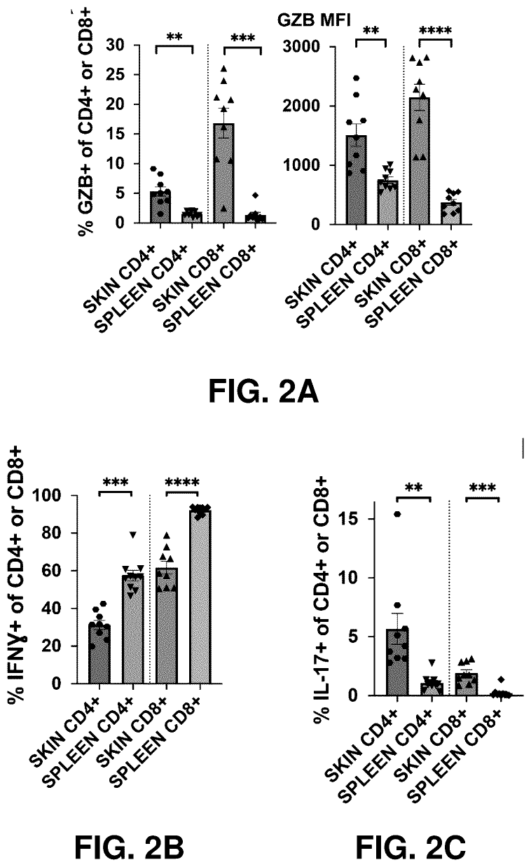

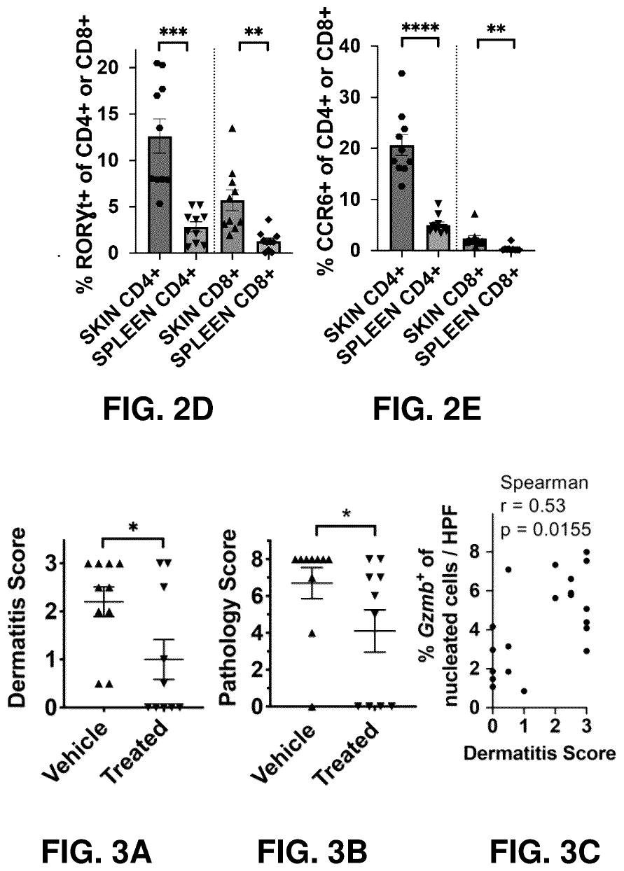

RNA sequencing experiments identified that both skin infiltrating CD4⁺ and CD8⁺ T cells from murine CLE possessed strong cytotoxic capacity with high Fasl and Gzmb expression (FIG. 1A, top panel). In addition, skin CD4⁺ T cells demonstrated a strong Th17 transcriptional signature, but not a Th1 phenotype (FIG. 1A, middle and bottom panels). These transcriptional results were further assessed by flow cytometric analyses, which confirmed T cell cytotoxic functionality and a dominant Th17 phenotype. As compared with splenic T cells, skin CD8+ and to a lesser extent CD4⁺ T cells expressed significantly more intracellular granzyme B than their splenic counterparts (FIG. 2A). In contrast, the percentage of IFN-γ⁺ skin CD4⁺ and CD8⁺ T cells after stimulation was significantly lower than that of splenocytes (FIG. 2B). Also observed was the dominant Th17 phenotype in murine CLE skin. A significantly greater fraction of skin-infiltrating CD4⁺ T cells was produced IL-17A after ex vivo stimulation as compared with splenic CD4⁺ T cells (FIG. 2C). Further, more skin-infiltrating CD4⁺ T cells expressed the canonical Th17 transcription factor RORγt (FIG. 2D) and Th17-associated chemokine receptor CCR6 (FIG. 2E) as compared with splenic T cells; in contrast, only a very small fraction of skin-infiltrating CD8⁺ T cells expressed IL-17A or Th17 phenotypic markers (FIGS. 2C-2E). These results further confirm the predominant Th17 phenotype in skin-infiltrating CD4⁺ T cells in murine CLE.

HIF-1α inhibition abrogates cutaneous disease in association with reduced cytotoxic T cell activity in diseased MRL/lpr skin.

To determine the effect of HIF-1 blockade on preventing skin disease, a 4-week treatment using a selective pharmacologic HIF-1α inhibitor was given to 16-week-old MRL/lpr mice. This treatment in younger (10-12-week-old) MRL/lpr mice decreases systemic disease, as measured by spleen size and anti-dsDNA production, and lupus nephritis, as measured by proteinuria, histopathology, and renal-infiltrating T cells (4). However, murine CLE skin lesions may not develop in MRL/lpr mice until after 14-16 weeks of age, and thus prior experiments did not allow for examination of the effect of HIF1 blockade on murine cutaneous lupus.

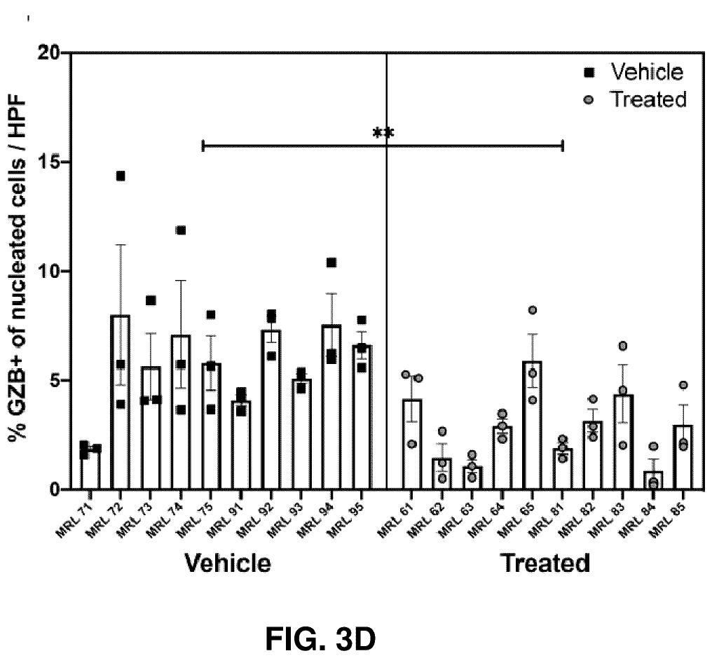

As compared with mice receiving PBS vehicle control, those who received PX-478, a selective HIF-1α inhibitor, had significantly reduced clinical skin disease (dermatitis score) at 20 weeks (FIG. 3A). In addition, histopathologic disease score (see Materials and Methods) of the interscapular skin was reduced in HIF-1α inhibitor-treated mice as compared with vehicle-treated mice (FIG. 3B) To identify a potential mechanism by which HIF-1 promotes pathogenic T cells in murine CLE, the expression of granzyme B in skin-infiltrating cells in PX-478 or vehicle-treated MRL/lpr mice was evaluated. Granzyme B expression was positively correlated with severity of clinical skin disease (dermatitis score) among both PX-478- and vehicle-treated mice (FIG. 3C), and PX-478 treatment not only decreased clinical skin disease severity and histopathologic disease score but also decreased the proportion of cells expressing granzyme B in affected skin (FIG. 3D). These data together indicated that HIF-1α inhibition may abrogate murine CLE by decreasing T cell cytotoxic activity.

HIF-1α Inhibition Reduces Cytotoxic Activity in CD8⁺ T Cells Isolated from MRL/Lpr CLE Skin.

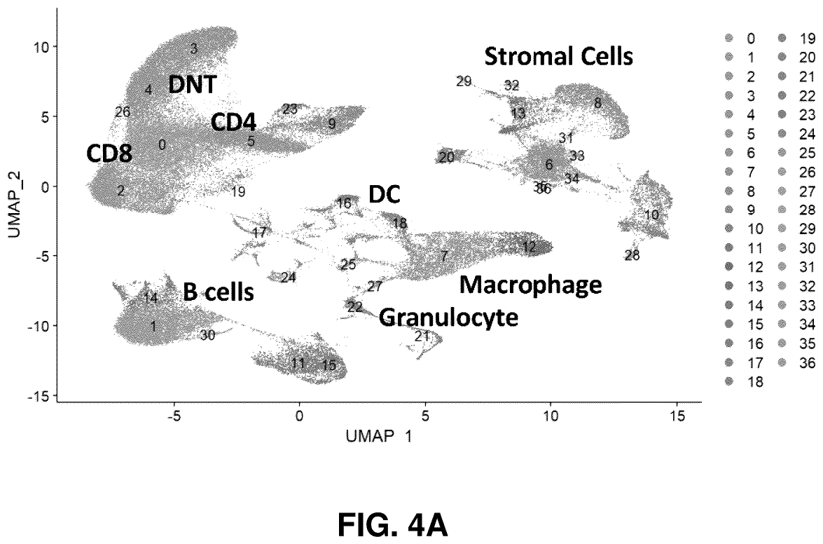

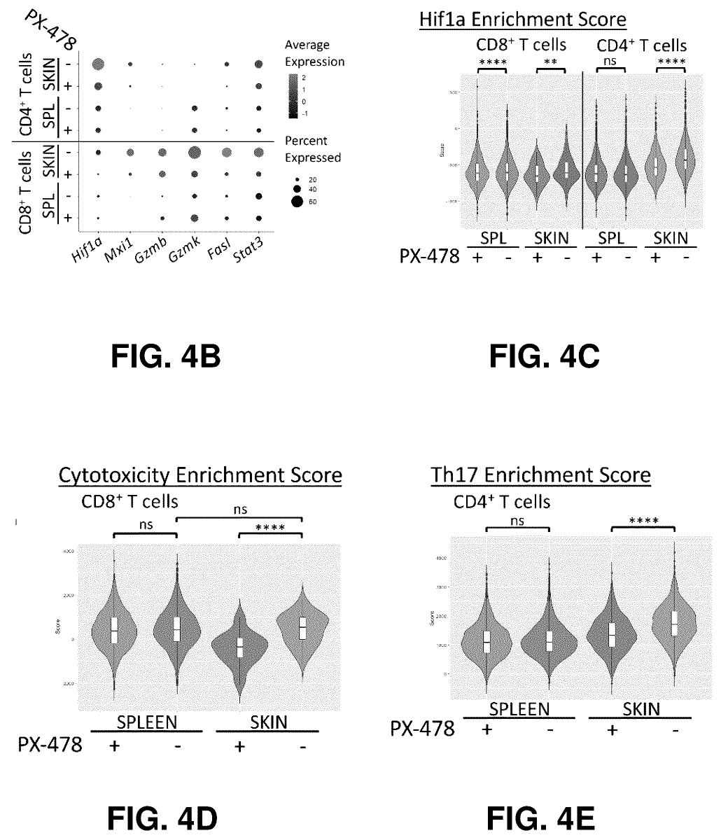

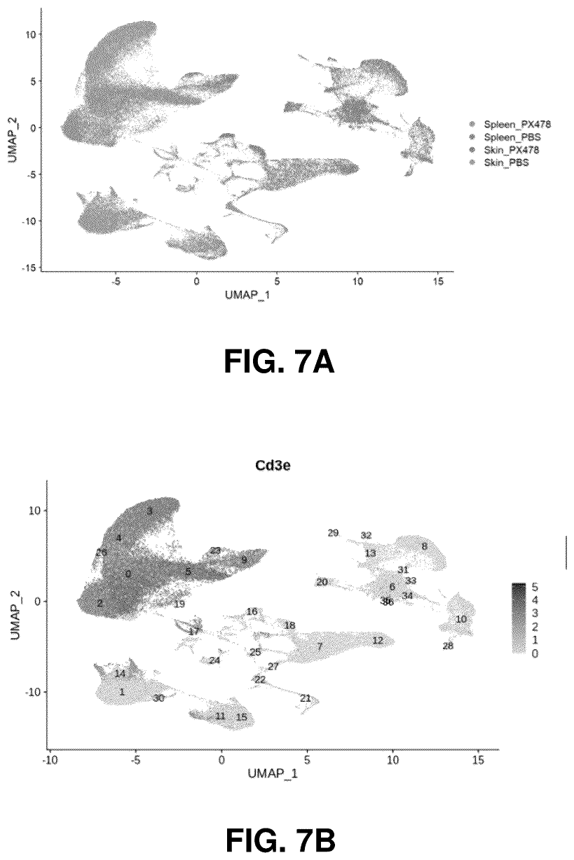

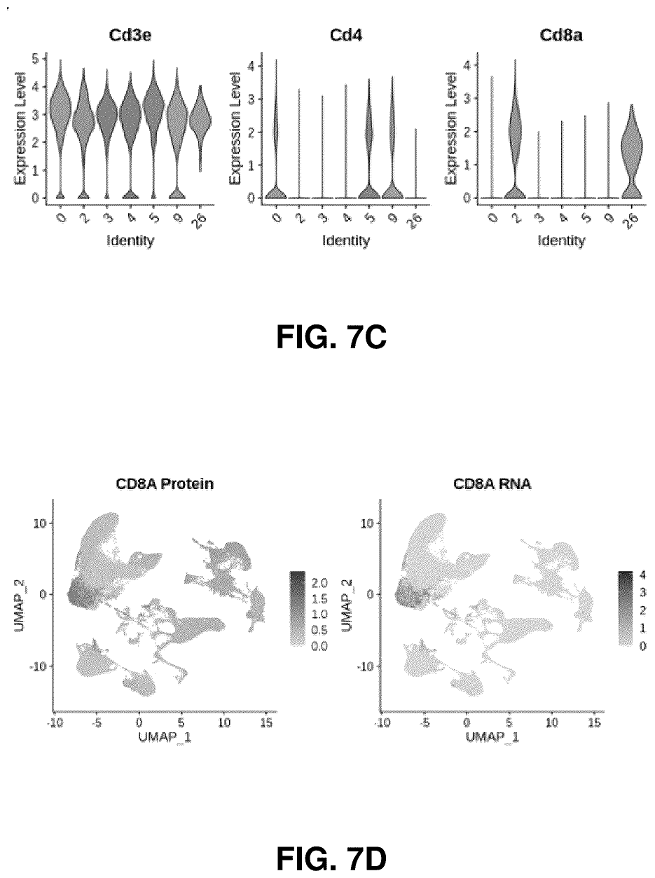

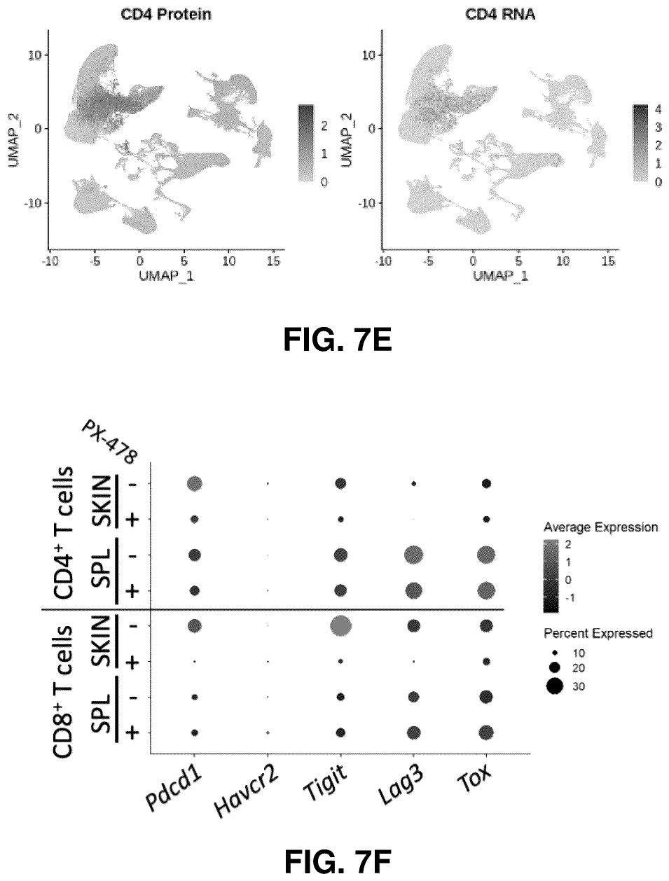

To confirm the effect of pharmacologic HIF-1α blockade on cytotoxic activity in skin-infiltrating T cells, performed was single-cell RNA sequencing of cells isolated from murine CLE skin and spleens taken simultaneously from MRL/lpr mice treated with a 5-day course of high dose PX-478 or PBS vehicle control. The single-cell suspension made from skin was enriched for hematopoietic (CD45+) cells, to allow for comparison of skin-infiltrating CD4⁺ and CD8⁺ T cells with their splenic counterparts. Clustering of spleen and skin cells revealed populations of CD4⁺ and CD8⁺ T cells from both spleen and skin, with an expanded double-negative T cell population identified only in splenocytes, and stromal cells including endothelial cells, fibroblasts and keratinocytes identified only in skin samples (FIG. 4A, FIG. 7A-7F). Selective pharmacologic HIF-1α inhibition decreased transcripts of Hif1a and Mxi1, a gene directly regulated by HIF-1α (19), in both CD4⁺ and CD8⁺ skin-infiltrating T cells but not splenic T cells (FIG. 4B). This was confirmed using GSEA, comparing gene expression in CD4⁺ and CD8⁺ T cell clusters against a previously identified set of genes controlled by HIF-1α (20). GSEA demonstrated that HIF-1 inhibitor treatment reduced Hif1α Enrichment Score in both CD4⁺ and CD8⁺ skin-infiltrating T cells (FIG. 4C). HIF-1 inhibition also reduced cytotoxic potential in CD8⁺ skin-infiltrating but not splenic T cells as demonstrated by decreased Gzmb, Gzmk, and Fasl transcripts (FIG. 4B) and reduced cytotoxicity enrichment score in skin-infiltrating CD8⁺ T cells (FIG. 4D), indicating an organ-specific effect of HIF-1 inhibition on T-cell cytotoxicity in the skin, supporting a causal role for HIF-1 in promoting pathogenic cytotoxic T cell activity in murine CLE. HIF-1 inhibition reduced expression of exhaustion markers Pdcd1 and Tigit in skin-infiltrating CD8⁺ T cells (FIG. 7F). HIF-1α inhibition also reduced Th17 activity in CD4⁺ T cells as demonstrated by a decrease in Th17 enrichment score in skin-infiltrating CD4⁺ T cells, which was not seen in splenic T cells (FIG. 4E), thus identifying a potential role for HIF-1 in promoting pathogenic Th17 T cell activity in murine cutaneous lupus.

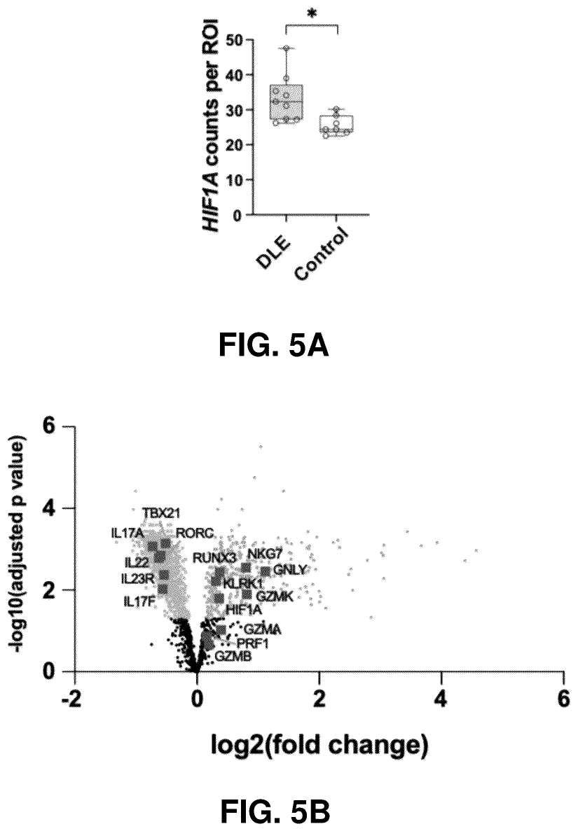

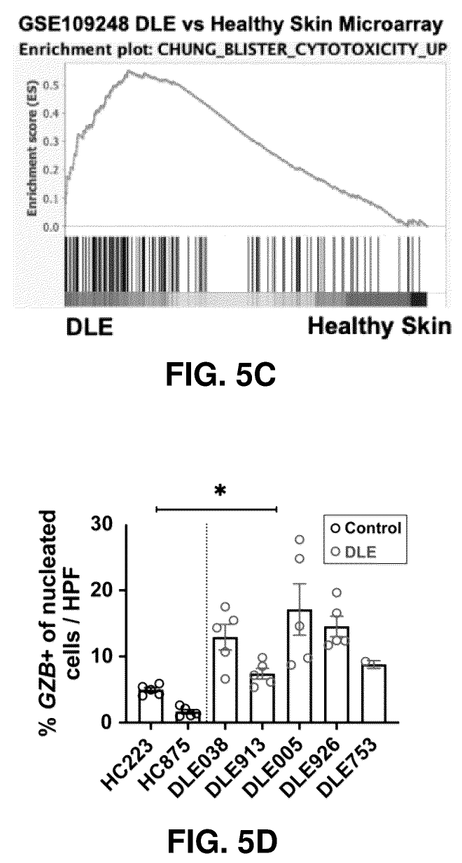

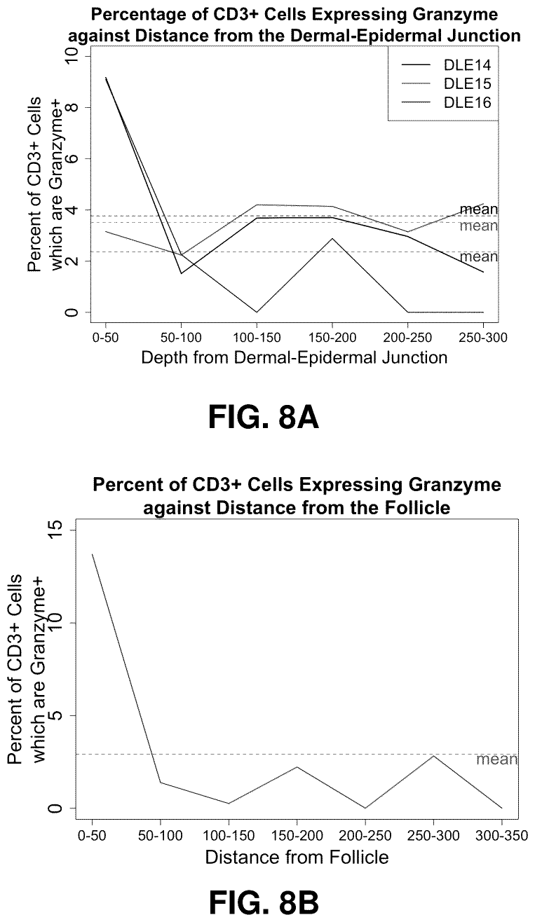

It was determined whether the HIF-1, Th17, and cytotoxic transcriptional signatures that were upregulated in murine CLE were similarly enhanced in skin biopsy specimens from patients with discoid lupus erythematosus (DLE), the most common subtype of CLE. The transcriptional profiles of regions of interest selected were analyzed to include T cell rich infiltrates using Nanostring GeoMx Digital Spatial Profiling, which identified upregulated HIF1A in DLE skin as compared with healthy control skin (FIG. 5A). The findings were in line with a recent microarray study of cutaneous lupus, which identified HIF1A network upregulation (21). The Nanostring data also revealed upregulation of cytotoxic signature molecules including NKG7, RUNX3, KLRK1, and GZMK (FIG. 5B), confirming that the cytotoxic signature identified in murine CLE-like skin is present in human DLE(22). Unlike murine cutaneous lupus, a Th17 signature in human DLE skin was not identified as compared with healthy skin. Instead, Th17-associated genes IL17A, IL22, IL23R, IL17F and RORC were more highly expressed in T cell-rich regions of healthy skin as compared with DLE skin (FIG. 5B), consistent with multiple prior studies that identify minimal Th17 activity in human DLE skin (22-24). Further confirmed was the robust cytotoxic signature found in DLE skin samples as compared with healthy control skin by performing GSEA, comparing gene signatures from a publicly available microarray dataset of 6 DLE and 14 healthy skin samples (25) against a human skin cytotoxicity signature generated from comparison of blister fluid to PBMCs in patients with Stevens-Johnson syndrome/toxic epidermal necrolysis (26) (FIG. 5C). Finally, skin biopsies from DLE patients for granzyme B expression by RNA in situ hybridization was evaluated, as previously done for MRL/lpr lesional skin (FIGS. 3A-3D). Human DLE skin lesions demonstrated elevated expression of GZMB RNA as compared with healthy human skin (FIG. 5D), with GZMB⁺ cells concentrated in the T cell rich inflammatory infiltrate. To specifically identify GZMB⁺ T cells in DLE lesional skin, immunofluorescence (IF) staining was performed for CD3 with concurrent RNA fluorescent in situ hybridization (FISH) to GZMB transcripts, identifying numerous GZMB⁺ T cells at the dermoepidermal junction and perifollicular regions. In the majority of cases analyzed, spatial quantification revealed enrichment of GZMB⁺ T cells at the DEJ and adjacent to the hair follicle (FIGS. 8A and 8B), which represent sites of skin tissue damage in DLE (27).

### Discussion

HIF-1 was identified as a key factor in generating pathogenic T cells in lupus skin disease in lupus-prone MRL/lpr mice, confirming it regulates effector function of skin tissue-infiltrating T cells. Cutaneous lupus skin-infiltrating T cells demonstrated increased effector function by transcript and protein expression as compared with their splenic counterparts. Cytotoxic effector function was decreased after systemic pharmacologic HIF-1 inhibition, which also ameliorated the development of skin lesions and skin histopathology score, indicating that pathogenic cytotoxic effector function may be a consequence of the upregulated HIF-1 signature in skin-infiltrating T cells. HIF-1 drives a transcriptional program that is linked to pathogenic features with enhanced T cell effector function (9,11), indicating it drives pathogenic T cell activity in cutaneous lupus. HIF-1 expression and cytotoxic effector capacity were also increased in human discoid lupus skin as compared with healthy skin, indicating the applicability of its therapeutic blockade to treat human discoid lupus.

Cytotoxic CD8⁺ T cells expressing granzyme B are substantially increased in cutaneous lupus skin biopsies (28-30), and drive the pathogenic process by promoting cellular apoptosis in the epidermis and papillary dermis (29), although the relative contribution of cytotoxic T cells to the development of tissue damage in skin is not known. Nor is it known whether cytotoxic CD4⁺ T cells, whose granzyme B secretion may be less tightly regulated as compared with their CD8⁺ counterparts (31), play a role in cutaneous lupus pathogenesis. A recent study using single-cell RNA sequencing of skin biopsies from lupus and healthy control patients did not find elevated activation, cytotoxicity or exhaustion profiles in T cells from lesional skin (24). However, these results may not accurately characterize the discoid lupus lesional T cell infiltrate due to the limited number of T and NK cells isolated from lesional skin (687 total cells); further, discoid lupus patients comprised only 3 of the 7 lupus subjects studied. Another recent study of single-cell RNA sequencing data from skin biopsies of patients with discoid lupus, systemic lupus erythematosus, or healthy controls captured over 40,000 dermal T cells, of which cytotoxic T lymphocytes were a major subset identified. The majority of cytotoxic T cells originated from DLE skin biopsies as compared with SLE or healthy control skin biopsies (32). Using multiple techniques, it was found that T cell rich areas of human DLE skin demonstrated upregulated HIF-1 and a cytotoxic signature, and the murine studies indicated that HIF-1 inhibition abrogated murine CLE by directly decreasing T cell cytotoxic activity, thus ameliorating tissue damage. These findings demonstrated that HIF-1 prevents exhaustion in CD8⁺ T cells and promotes granzyme B production, and reduced granzyme B production after treatment with HIF-1a shRNA. It is also possible that prevention of tissue damage in the setting of HIF-1 inhibition is due to reduced T cell survival in the skin microenvironment.

In addition to CD8⁺ T cells, certain subsets of CD4⁺ T cells, including Th1 and Th17 cells, are increased in some studies of human DLE skin (6). The present studies did not identify a strong Th1 signature in mouse or human DLE skin; however, a strong Th17 signature was notable at the transcript and protein level in murine cutaneous lupus skin. The role of IL-17 in human DLE pathogenesis is controversial. IL-17 producing T cells have been identified in kidney and skin lesions of lupus patients (33-35), with one study of DLE patients identifying IL-17A as highly expressed in both serum and damaged skin tissue (36). However, subsequent studies of lesional DLE skin using microarray transcriptomic analyses or T cell crawlout methods quantifying IL-17A production after T-cell stimulation did not identify a dominant Th17 profile (22, 23). Further, Th17 cells were not identified in T cell subset analyses from single-cell RNA sequencing studies of DLE skin (24,32). Similarly, the present human DLE transcriptomic analyses did not identify a Th17 signature compared with healthy skin. In fact, T-cell rich areas of DLE skin biopsies expressed somewhat lower Th17 marker gene expression than healthy skin. The cause of this discrepancy between the murine model of cutaneous lupus and human skin disease is not clear, but could involve differences in skin commensals, which may alter T-cell IL-17 production (37, 38). Nonetheless, if present, it is possible that even low amounts of IL-17 may contribute to skin tissue damage in DLE. If so, this may be driven in part by HIF-1, as it favors pathogenic Th17 differentiation by targeting the proteosomal degradation of Foxp3 to control the balance between Th17 and Treg cells (11).

The factors driving HIF-1 upregulation in DLE skin have not been fully elucidated. Unlike what was observed in murine lupus nephritis (4), its upregulation in skin-infiltrating T cells was not related to detectable local tissue hypoxia as measured by pimonidazole staining, which can detect moderate to severely hypoxic conditions with oxygen tensions below 10 mmHg (18). However, pimonidazole does not identify modest to moderate hypoxic conditions, with oxygen tensions between 10-19 mmHg (14), which may promote HIF-1 expression. Such conditions are likely present in lesional cutaneous lupus skin due to tissue damage and increased metabolic demands of the inflammatory infiltrate, though further studies will be required to characterize the oxygen tension in inflamed skin. In addition, hypoxia-independent mechanisms may increase HIF-1 expression in CLE skin-infiltrating T cells. Toll-like receptor and T cell receptor signaling stabilize HIF-1 under normoxic conditions (7, 8), and T cell activation further enhances its stabilization in T cells cultured under hypoxic conditions (8), indicating the potential for synergy between hypoxia-dependent and independent mechanisms. HIF-1 also accumulates in skin keratinocytes in response to ultraviolet (UV) light, which is mediated by mitochondrial reactive oxygen species (mROS) (16). The metabolic stress signal mROS is induced upon T cell activation (8), and upregulates HIF-1 in lymphocytes (39).

Taken together, these results indicate that increased HIF-1 expression in CLE skin-infiltrating T cells promotes skin tissue damage, and its inhibition abrogates development of disease. It is possible that skin-infiltrating T cells upregulate HIF-1 after infiltrating into the skin, resulting in reprogramming of the T cells to subtypes with heightened effector functions, ultimately causing tissue damage. The identification of HIF-1 as a novel therapeutic target for DLE is useful particularly because selective inhibitors of HIF-1 are available and have been well-tolerated in phase II clinical trials for cancers (40).

### REFERENCES

1. Tsokos G C. Systemic lupus erythematosus. N Engl J Med. 2011; 365(22):2110-21.
2. Lee L A, Gaither K K, Coulter S N, Norris D A, and Harley J B. Pattern of cutaneous immunoglobulin G deposition in subacute cutaneous lupus erythematosus is reproduced by infusing purified anti-Ro (SSA) autoantibodies into human skin-grafted mice. J Clin Invest. 1989; 83(5):1556-62.
3. Billi A C, Ma F, Plazyo O, Gharaee-Kermani M, Wasikowski R, Hile G A, et al. Nonlesional lupus skin contributes to inflammatory education of myeloid cells and primes for cutaneous inflammation. Sci Transl Med. 2022; 14(642):eabn2263.
4. Chen P-M, Wilson P C, Shyer J A, Veselits M, Steach H R, Cui C, et al. Kidney tissue hypoxia dictates T cell-mediated injury in murine lupus nephritis. Sci Transl Med. 2020; 12(538).
5. Little A J, and Vesely M D. Cutaneous Lupus Erythematosus: Current and Future Pathogenesis-Directed Therapies. Yale J Biol Med. 2020; 93(1):81-95.
6. Achtman J C, and Werth V P. Pathophysiology of cutaneous lupus erythematosus. Arthritis Res Ther. 2015; 17:182.
7. Tao J H, Barbi J, and Pan F. Hypoxia-inducible factors in T lymphocyte differentiation and function. A Review in the Theme: Cellular Responses to Hypoxia. Am J Physiol Cell Physiol. 2015; 309(9):C₅₈₀-9.
8. Phan A T, and Goldrath A W. Hypoxia-inducible factors regulate T cell metabolism and function. Mol Immunol. 2015; 68(2 Pt C):527-35.
9. Doedens A L, Phan A T, Stradner M H, Fujimoto J K, Nguyen J V, Yang E, et al. Hypoxia-inducible factors enhance the effector responses of CD8(+) T cells to persistent antigen. Nat Immunol. 2013; 14(11):1173-82.
10. Liikanen I, Lauhan C, Quon S, Omilusik K, Phan A T, Bartroli L B, et al. Hypoxia-inducible factor activity promotes antitumor effector function and tissue residency by CD8+ T cells. J Clin Invest. 2021; 131(7).
11. Dang E V, Barbi J, Yang H Y, Jinasena D, Yu H, Zheng Y, et al. Control of T(H)17/T(reg) balance by hypoxia-inducible factor 1. Cell. 2011; 146(5):772-84.
12. Shi L Z, Wang R, Huang G, Vogel P, Neale G, Green D R, et al. HIF1α-dependent glycolytic pathway orchestrates a metabolic checkpoint for the differentiation of TH17 and Treg cells. J Exp Med. 2011; 208(7):1367-76.
13. Rezvani H R, Ali N, Nissen L J, Harfouche G, de Verneuil H, Taieb A, et al. HIF-1alpha in epidermis: oxygen sensing, cutaneous angiogenesis, cancer, and non-cancer disorders. J Invest Dermatol. 2011; 131(9):1793-805.
14. Evans S M, Schrlau A E, Chalian A A, Zhang P, and Koch C J. Oxygen levels in normal and previously irradiated human skin as assessed by EF5 binding. J Invest Dermatol. 2006; 126(12):2596-606.
15. Meller S, Winterberg F, Gilliet M, Muller A, Lauceviciute I, Rieker J, et al. Ultraviolet radiation-induced injury, chemokines, and leukocyte recruitment: An amplification cycle triggering cutaneous lupus erythematosus. Arthritis Rheum. 2005; 52(5):1504-16.
16. Rezvani H R, Dedieu S, North S, Belloc F, Rossignol R, Letellier T, et al. Hypoxia-inducible factor-1alpha, a key factor in the keratinocyte response to UVB exposure. J Biol Chem. 2007; 282(22):16413-22.
17. Clever D, Roychoudhuri R, Constantinides M G, Askenase M H, Sukumar M, Klebanoff C A, et al. Oxygen Sensing by T Cells Establishes an Immunologically Tolerant Metastatic Niche. Cell. 2016; 166(5):1117-31 e14.
18. Rosenberger C, Rosen S, Paliege A, and Heyman S N. Pimonidazole adduct immunohistochemistry in the rat kidney: detection of tissue hypoxia. Methods Mol Biol. 2009; 466:161-74.
19. Lofstedt T, Fredlund E, Noguera R, Navarro S, Holmquist-Mengelbier L, Beckman S, et al. HIF-1alpha induces MXI1 by alternate promoter usage in human neuroblastoma cells. Exp Cell Res. 2009; 315(11):1924-36.
20. Zhang H, Li H, Xi H S, and Li S. HIF1alpha is required for survival maintenance of chronic myeloid leukemia stem cells. Blood. 2012; 119(11):2595-607.
21. Berthier C C, Tsoi L C, Reed T J, Stannard J N, Myers E M, Namas R, et al. Molecular Profiling of Cutaneous Lupus Lesions Identifies Subgroups Distinct from Clinical Phenotypes. J Clin Med. 2019; 8(8).
22. Jabbari A, Suarez-Farinas M, Fuentes-Duculan J, Gonzalez J, Cueto I, Franks A G, Jr., et al. Dominant Th1 and minimal Th17 skewing in discoid lupus revealed by transcriptomic comparison with psoriasis. J Invest Dermatol. 2014; 134(1):87-95.
23. Dey-Rao R, Smith J R, Chow S, and Sinha A A. Differential gene expression analysis in CCLE lesions provides new insights regarding the genetics basis of skin vs. systemic disease. Genomics. 2014; 104(2):144-55.
24. Dunlap G S, Billi A C, Xing X, Ma F, Maz M P, Tsoi L C, et al. Single-cell transcriptomics reveals distinct effector profiles of infiltrating T cells in lupus skin and kidney. JCI Insight. 2022; 7(8).
25. Mande P, Zirak B, Ko W C, Taravati K, Bride K L, Brodeur T Y, et al. Fas ligand promotes an inducible TLR-dependent model of cutaneous lupus-like inflammation. J Clin Invest. 2018; 128(7):2966-78.
26. Chung W H, Hung S I, Yang J Y, Su S C, Huang S P, Wei C Y, et al. Granulysin is a key mediator for disseminated keratinocyte death in Stevens-Johnson syndrome and toxic epidermal necrolysis. Nat Med. 2008; 14(12):1343-50.
27. Saenz-Corral C I, Vega-Memije M E, Martinez-Luna E, Cuevas-Gonzalez J C, Rodriguez-Carreon A A, de la Rosa J J, et al. Apoptosis in chronic cutaneous lupus erythematosus, discoid lupus, and lupus profundus. Int J Clin Exp Pathol. 2015; 8(6):7260-5.
28. Wenzel J, Uerlich M, Worrenkamper E, Freutel S, Bieber T, and Tuting T. Scarring skin lesions of discoid lupus erythematosus are characterized by high numbers of skin-homing cytotoxic lymphocytes associated with strong expression of the type I interferon-induced protein MxA. Br J Dermatol. 2005; 153(5):1011-5.
29. Grassi M, Capello F, Bertolino L, Seia Z, and Pippione M. Identification of granzyme B-expressing CD-8-positive T cells in lymphocytic inflammatory infiltrate in cutaneous lupus erythematosus and in dermatomyositis. Clin Exp Dermatol. 2009; 34(8):910-4.
30. Fogagnolo L, Soares T C, Senna C G, Souza E M, Blotta M H, and Cintra M L. Cytotoxic granules in distinct subsets of cutaneous lupus erythematosus. Clin Exp Dermatol. 2014; 39(7):835-9.
31. Lin L, Couturier J, Yu X, Medina M A, Kozinetz C A, and Lewis D E. Granzyme B secretion by human memory CD4 T cells is less strictly regulated compared to memory CD8 T cells. BMC Immunol. 2014; 15:36.
32. Zheng M, Hu Z, Mei X, Ouyang L, Song Y, Zhou W, et al. Single-cell sequencing shows cellular heterogeneity of cutaneous lesions in lupus erythematosus. Nat Commun. 2022; 13(1):7489.
33. Crispin J C, and Tsokos G C. Interleukin-17-producing T cells in lupus. Curr Opin Rheumatol. 2010; 22(5):499-503.
34. Crispin J C, Oukka M, Bayliss G, Cohen R A, Van Beek C A, Stillman I E, et al. Expanded double negative T cells in patients with systemic lupus erythematosus produce IL-17 and infiltrate the kidneys. J Immunol. 2008; 181(12):8761-6.
35. Oh S H, Roh H J, Kwon J E, Lee S H, Kim J Y, Choi H J, et al. Expression of interleukin-17 is correlated with interferon-alpha expression in cutaneous lesions of lupus erythematosus. Clin Exp Dermatol. 2011; 36(5):512-20.
36. Tanasescu C, Balanescu E, Balanescu P, Olteanu R, Badea C, Grancea C, et al. IL-17 in cutaneous lupus erythematosus. Eur J Intern Med. 2010; 21(3):202-7.
37. Harrison O J, Linehan J L, Shih H Y, Bouladoux N, Han S J, Smelkinson M, et al. Commensal-specific T cell plasticity promotes rapid tissue adaptation to injury. Science. 2019; 363(6422).
38. Hurabielle C, Link V M, Bouladoux N, Han S J, Merrill E D, Lightfoot Y L, et al. Immunity to commensal skin fungi promotes psoriasiform skin inflammation. Proc Natl Acad Sci USA. 2020; 117(28):16465-74.
39. Khan H, Anshu A, Prasad A, Roy S, Jeffery J, Kittipongdaja W, et al. Metabolic Rewiring in Response to Biguanides Is Mediated by mROS/HIF-1α in Malignant Lymphocytes. Cell Rep. 2019; 29(10):3009-18 e4.
40. Fallah J, and Rini B I. HIF Inhibitors: Status of Current Clinical Development. Curr Oncol Rep. 2019; 21(1):6.
41. Broggi A, Cigni C, Zanoni I, and Granucci F. Preparation of Single-cell Suspensions for Cytofluorimetric Analysis from Different Mouse Skin Regions. J Vis Exp. 2016(110):e52589.
42. Scharping N E, Menk A V, Whetstone R D, Zeng X, and Delgoffe G M. Efficacy of PD-1 Blockade Is Potentiated by Metformin-Induced Reduction of Tumor Hypoxia. Cancer immunology research. 2017; 5(1):9-16.
43. Chan E S, Herlitz L C, and Jabbari A. Ruxolitinib Attenuates Cutaneous Lupus Development in a Mouse Lupus Model. J Invest Dermatol. 2015; 135(7):1912-5.
44. Chan O T, Paliwal V, McNiff J M, Park S H, Bendelac A, and Shlomchik M J. Deficiency in beta(2)-microglobulin, but not CD1, accelerates spontaneous lupus skin disease while inhibiting nephritis in MRL-Fas(lpr) nice: an example of disease regulation at the organ level. J Immunol. 2001; 167(5):2985-90.
45. Love M I, Huber W, and Anders S. Moderated estimation of fold change and dispersion for RNA-seq data with DESeq2. Genome Biol. 2014; 15(12):550.
46. Ekiz H A, Conley C J, Stephens W Z, and O'Connell R M. CIPR: a web-based R/shiny app and R package to annotate cell clusters in single cell RNA sequencing experiments. BMC Bioinformatics. 2020; 21(1):191.
47. Borcherding N, Vishwakarma A, Voigt A P, Bellizzi A, Kaplan J, Nepple K, et al. Mapping the immune environment in clear cell renal carcinoma by single-cell genomics. Commun Biol. 2021; 4(1):122.
48. Kanehisa M, Furumichi M, Sato Y, Ishiguro-Watanabe M, and Tanabe M. KEGG: integrating viruses and cellular organisms. Nucleic Acids Res. 2021; 49(D1):D545-D51.
49. Blake J A, Baldarelli R, Kadin J A, Richardson J E, Smith C L, Bult C J, et al. Mouse Genome Database (MGD): Knowledgebase for mouse-human comparative biology. Nucleic Acids Res. 2021; 49(D1):D981-D7.
50. Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA. 2005; 102(43):15545-50.
51. Wang A, Fogel A L, Murphy M J, Panse G, McGeary M K, McNiff J M, et al. Cytokine RNA In Situ Hybridization Permits Individualized Molecular Phenotyping in Biopsies of Psoriasis and Atopic Dermatitis. JID Innov. 2021; 1(2):100021.
52. Merritt C R, Ong G T, Church S E, Barker K, Danaher P, Geiss G, et al. Multiplex digital spatial profiling of proteins and RNA in fixed tissue. Nat Biotechnol. 2020; 38(5):586-99.
53. Little, “Pathogenic T cells in discoid lupus erythematosus,” NIH project number 1K08AR081408-01A1, start date Jun. 1, 2023.
54. Craft, “Pathogenesis of Lupus Nephritis,” NIH project number 5R01AI152443-05, start date May 6, 2020.
55. Little, et al., “HIF-1 regulates pathogenic cytotoxic T cells in lupus skin disease,” JCI Insight, 10.1172/jci.insight.166076, 18 pages.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein Those skilled in the art will recognize, or be able to ascertain, using no more than routine experimentation, many equivalents to the specific embodiments of the method and compositions described herein. Such equivalents are intended to be encompassed by the following claims.

We claim:

1. A method of treating an autoimmune skin disease or disorder in a subject comprising administering the subject an effective amount of a hypoxia-inducible factor-1 (HIF-1) inhibitor.

2. The method of claim 1, wherein the autoimmune disease is T cell-mediated.

3. The method of claim 2, wherein the T cells are skin-infiltrating T cells.

4. The method of claim 1, wherein the HIF-1 inhibitor is administered in an effective amount to reduce the HIF-1 expression signature, reduce the cytotoxic activity, reduce expression of exhaustion marker(s), reduce Th17 phenotype, and/or reduce intracellular granzyme B in CD4+ and/or CD8+ T cells.

5. The method of claim 1, wherein the subject does not have lupus nephritis.

6. The method of claim 1, wherein the subject does not have cancer.

7. The method of claim 1, wherein the subject does not have an angiogenic disease or disorder.

8. The method of claim 1, wherein the subject does not have psoriasis, scleroderma, or pyogenic granulomas.

9. The method of claim 1, wherein the subject has cutaneous lupus, pemphigus, pemphigoid, epidermolysis bullosa acquisita vitiligo, lichen planus, lichen sclerosus, dermatomyositis, alopecia areata, or Sjögren's syndrome.

10. The method of claim 1, wherein the subject has cutaneous lupus selected from discoid cutaneous lupus, subacute cutaneous lupus, and acute cutaneous lupus.

11. The method of claim 1, wherein the subject does not have systemic lupus erythematosus (SLE).

12. The method of claim 1, wherein the HIF-1 inhibitor is a small molecule, functional nucleic acid, or inhibitory polypeptide or protein.

13. The method of claim 1, wherein the HIF-1 inhibitor is selected from PX-478, chemotin, topotecan, 103D5R, YC-1, GL331, geldanamycin, 2-ME2, bisphenol, berberine, and PX-12, or a pharmaceutically acceptable salt thereof.

14. The method of claim 1, wherein the HIF-1 inhibitor is a functional nucleic acid selected from antisense molecules, siRNA, miRNA, aptamers, ribozymes, RNAi, and external guide sequences.

15. The method of claim 13, wherein the functional nucleic acid targets a segment of a nucleic acid encoding the amino acid sequence of SEQ ID NO:1, or the complement thereof, or variants thereof having a nucleic acid sequence at least 65%, 70%, 75%, 80%, 85%, 90%, or 95% identical to a nucleic acid encoding the amino acid sequence of SEQ ID NO:1.

16. The method of claim 15, wherein the functional nucleic acid targets a segment of the nucleic acid sequence of SEQ ID NO:2, or the complement thereof, or a genomic sequence corresponding therewith, or variants thereof having a nucleic acid sequence at least 65%, 70%, 75%, 80%, 85%, 90%, or 95% identical to SEQ ID NO:2.

17. The method of claim 1, wherein the HIF-1 inhibitor is an inhibitor polypeptide comprising the amino acid sequence of any one of SEQ ID NOS:5-8, or variant thereof having a nucleic acid sequence at least 65%, 70%, 75%, 80%, 85%, 90%, or 95% identical to SEQ ID NO:5-8, or a nucleic acid encoding the same, optionally wherein the HIF-1 inhibitor has a structure selected from:

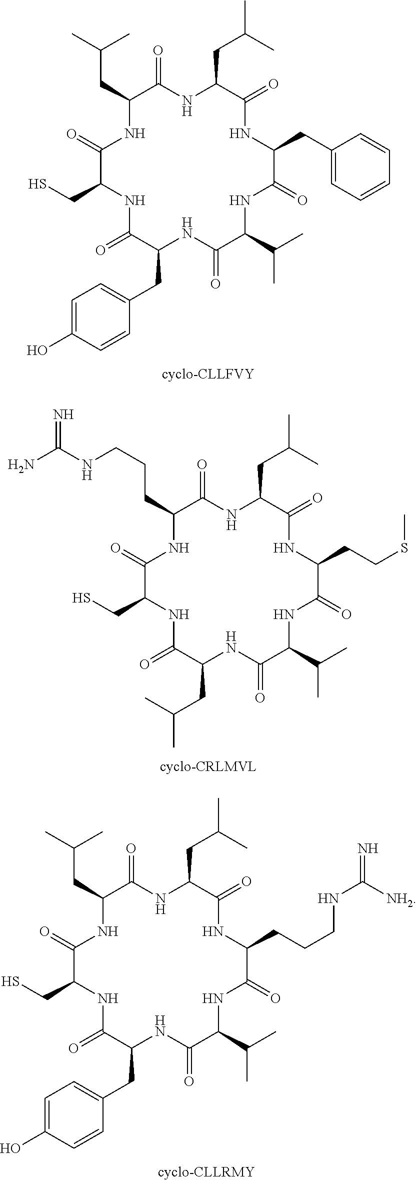 [SMILES: CC(C)C[C@@H]1NC(=O)[C@H](CS)NC(=O)[C@H](Cc2ccc(O)cc2)NC(=O)[C@H](C(C)C)NC(=O)[C@H](Cc2ccccc2)NC(=O)[C@H](CC(C)C)NC1=O ; CSCC[C@@H]1NC(=O)[C@H](CC(C)C)NC(=O)[C@H](CCCNC(=N)N)NC(=O)[C@H](CS)NC(=O)[C@H](CC(C)C)NC(=O)[C@H](C(C)C)NC1=O ; CC(C)C[C@@H]1NC(=O)[C@H](CS)NC(=O)[C@H](Cc2ccc(O)cc2)NC(=O)[C@H](C(C)C)NC(=O)[C@H](CCCNC(=N)N)NC(=O)[C@H](CC(C)C)NC1=O]

18. The method of claim 17, wherein the HIF-1 inhibitor further comprises a protein transduction domain.

19. The method of claim 1, wherein the HIF-1 inhibitor is in a pharmaceutical composition further comprising a carrier.

20. The method of claim 1, wherein the HIF-1 inhibitor is administered systemically.

21. The method of claim 1, wherein the HIF-1 inhibitor is administered locally to skin effected by the autoimmune disease.

22. The method of claim 21, wherein the HIF-1 inhibitor is administered by topical administration, injection, or intralesional administration.

23. The method of claim 22, wherein the HIF-1 inhibitor is administered by topical administration in a pharmaceutical composition suitable for topical administration.

24. The method of claim 23, wherein the pharmaceutical composition comprises a penetration enhancer.

25. A pharmaceutical composition comprising a HIF-1 inhibitor and a penetration enhancer.

26. The pharmaceutical composition of claim 25, wherein the composition is a lotion or cream.

27. A patch comprising the pharmaceutical composition a HIF-1 inhibitor and optionally and a penetration enhancer.


SEQUENCE LISTING (17 sequences)

SEQ ID NO: 1 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 2 - RNA, 3678 nt, Homo sapiens
  source 1..3678: mol_type = mRNA
gtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtgga

SEQ ID NO: 3 - protein, 12 aa, synthetic construct
MEGAGGANDKKK

SEQ ID NO: 4 - protein, 36 aa, synthetic construct
MSSQCRSLENKFVFLKEGLGNSKPEELEEIRIENGR

SEQ ID NO: 5 - protein, 349 aa, Homo sapiens
MAATAAEAVASGSGEPREEAGALGPAWDESQLRSYSFPTRPIPRLSQSDPRAEELIENEEPVVLTDTNLVYPALKWDLEYLQENIGNGDFSVYSASTHKFLYYDEKKMANFQNFKPRSNREEMKFHEFVEKLQDIQQRGGEERLYLQQTLNDTVGRKIVMDFLGFNWNWINKQQGKRGWGQLTSNLLLIGMEGNVTPAHYDEQQNFFAQIKGYKRCILFPPDQFECLYPYPVHHPCDRQSQVDFDNPDYERFPNFQNVVGYETVVGPGDVLYIPMYWWHHIESLLNGGITITVNFWYKGAPTPKRIEYPLKAHQKVAIMRNIEKMLGEALGNPQEVGPLLNTMIKGRYN

SEQ ID NO: 6 - protein, 6 aa, synthetic construct
  SITE 1..6: note = cysteine tyrosine thioester (Cys-Tyr)
CLLFVY

SEQ ID NO: 7 - protein, 6 aa, synthetic construct
  SITE 1..6: note = Cysteine Leucine Thioester (Cys-Leu)
CRLMVL

SEQ ID NO: 8 - protein, 6 aa, synthetic construct
  SITE 1..6: note = Cysteine Tyrosine Thioester (Cys-Tyr)
CLLRMY

SEQ ID NO: 9 - protein, 7 aa, synthetic construct
RRRRRRR

SEQ ID NO: 10 - protein, 12 aa, synthetic construct
RRQRRTSKLMKR

SEQ ID NO: 11 - protein, 27 aa, synthetic construct
GWTLNSAGYLLGKINLKALAALAKKIL

SEQ ID NO: 12 - protein, 29 aa, synthetic construct
WEAKLAKALAKALAKHLAKALAKALKCEA

SEQ ID NO: 13 - protein, 16 aa, synthetic construct
RQIKIWFQNRRMKWKK

SEQ ID NO: 14 - protein, 20 aa, synthetic construct
GDIMGEWGNEIFGAIAGFLG

SEQ ID NO: 15 - protein, 35 aa, synthetic construct
RRRRRRRRRRRGEGDIMGEWGNEIFGAIAGFLGGE

SEQ ID NO: 16 - protein, 11 aa, synthetic construct
YGRKKRRQRRR

SEQ ID NO: 17 - protein, 9 aa, synthetic construct
RKKRRQRRR